SEQUENCE LISTING (12 sequences)

SEQ ID NO: 1 - protein, 554 aa, synthetic construct
MFSFVDLRLLLLLAATALLTHGQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNSEFYYVLCAVSVVGDPILNSTYWSGSLMMTRLAVKPKNNGESYNQHQFALRNIEKGMYDKVMPYGPSGIKQGDTLYFPAVGFLVRTEFKYNDSNCPVAKCQYSKPENCRLSMGIRPNSHYILRSGLLKYNLSDEENSKIIFIEISDQRLSIGSPSKIYDSLGQPVFYQASFSWDTMIKFGDVQTVNPLVVNWRDNTVISRPGQSQCPRFNTCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 2 - RNA, 1662 nt, synthetic construct
  source 1..1662: mol_type = other RNA
atgttctctttcgtggacctgcgcctgctgctgctgctcgctgccaccgccctgctgacacacggccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacc

SEQ ID NO: 3 - RNA, 1950 nt, synthetic construct
  source 1..1950: mol_type = other RNA
aggccggcactcttctggtccccacagactcagagagaacccgccgccaccatgttctctttcgtggacctgcgcctgctgctgctgctcgctgccaccgccctgctgacacacggccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacctgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 4554 bp, synthetic construct
  source 1..4554: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgttctctttcgtggacctgcgcctgctgctgctgctcgctgccaccgccctgctgacacacggccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacctgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 5 - protein, 602 aa, synthetic construct
MPTESKKVRFENTASDKGKNPSKVIKSYYGTMDIKKINEGLLDSKILSAFNTVIALLGSIVIIVMNIMIIQNYTRSTDNQAMIKDALQSIQQQIKGLADKIGTEIGPKVSLIDTSSTITIPANIGLLGSKISQSTASINENVNEKCKFTLPPLKIHECNISCPNPLPFREYKPQTEGVSNLVGLPNNICLQKTSNQILKPKLISYTLPVVGQSGTCITDPLLAMDEGYFAYSHLEKIGSCSRGVSKQRIIGVGEVLDRGDEVPSLFMTNVWTPSNPNTVYHCSAVYNSEFYYVLCAVSVVGDPILNSTYWSGSLMMTRLAVKPKNNGESYNQHQFALRNIEKGMYDKVMPYGPSGIKQGDTLYFPAVGFLVRTEFKYNDSNCPVAKCQYSKPENCRLSMGIRPNSHYILRSGLLKYNLSDEENSKIIFIEISDQRLSIGSPSKIYDSLGQPVFYQASFSWDTMIKFGDVQTVNPLVVNWRDNTVISRPGQSQCPRFNTCPEVCWEGVYNDAFLIDRINWISAGVFLDSNQTAENPVFTVFKDNEVLYRAQLASEDTNAQKTITNCFLLKNKIWCISLVEIYDTGDNVIRPKLFAVKIPEQCT

SEQ ID NO: 6 - RNA, 1806 nt, synthetic construct
  source 1..1806: mol_type = other RNA
atgcctacagaaagcaaaaaggtgcggttcgagaacacagcctctgacaagggaaagaatcctagcaaggtgatcaaaagctattacggcaccatggatatcaagaagattaacgaaggcctgctggacagcaagatactcagcgcttttaacaccgtgatcgccctgctgggcagcatcgtgatcatagttatgaacatcatgattatccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacc

SEQ ID NO: 7 - RNA, 2094 nt, synthetic construct
  source 1..2094: mol_type = other RNA
aggccggcactcttctggtccccacagactcagagagaacccgccgccaccatgcctacagaaagcaaaaaggtgcggttcgagaacacagcctctgacaagggaaagaatcctagcaaggtgatcaaaagctattacggcaccatggatatcaagaagattaacgaaggcctgctggacagcaagatactcagcgcttttaacaccgtgatcgccctgctgggcagcatcgtgatcatagttatgaacatcatgattatccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacctgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 4698 bp, synthetic construct
  source 1..4698: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatgcctacagaaagcaaaaaggtgcggttcgagaacacagcctctgacaagggaaagaatcctagcaaggtgatcaaaagctattacggcaccatggatatcaagaagattaacgaaggcctgctggacagcaagatactcagcgcttttaacaccgtgatcgccctgctgggcagcatcgtgatcatagttatgaacatcatgattatccagaactacacccggagcaccgacaaccaggctatgatcaaagacgccctgcagagcatccaacagcagatcaagggcctggccgacaagatcggcacagaaatcggaccaaaggtgtccctgatcgacacgagttctacaatcactatccctgccaacatcggcctgctgggcagcaaaatctctcagagcacagccagcatcaacgagaacgtgaacgagaaatgcaagttcaccctgccaccactgaagatccacgaatgcaacatcagctgccctaatcctctgcccttcagagagtacaagcctcagaccgagggtgtgtctaacctggtgggcctgccgaacaacatctgcctgcaaaaaaccagcaaccagatcctgaaacctaagctgatcagctacacactgcctgtggtgggccagagcggcacctgtatcacagatcctctgctggccatggatgaaggctacttcgcctacagccatctggaaaagatcgggagctgtagccggggcgtctccaaacagagaatcatcggcgtgggcgaagtgctggacagaggcgatgaggtcccctccctgtttatgacaaatgtgtggacccctagcaaccctaacacagtgtaccactgctccgccgtgtataattctgaattctactacgtgctgtgcgccgtgtccgtggtgggagaccccatcctgaactctacctactggagcggctctctgatgatgaccagactggctgttaagcccaagaacaacggcgagagctacaatcaacaccagttcgccctgcggaacatcgagaagggcatgtacgacaaagtgatgccctacggcccttcaggaatcaagcagggcgataccctgtatttccccgctgtgggcttcctggtgcggaccgaattcaagtacaatgactccaattgccccgtggccaagtgtcagtacagcaaacctgaaaactgtagactgtctatgggcatcagacctaatagccactacatcctcagaagcggactcctcaagtacaacctgtccgacgaggaaaactctaaaattatcttcatcgagatcagcgaccagcgcctgtctatcggatctccatctaagatctacgatagcctgggccaacctgtgttttaccaggccagctttagctgggacaccatgatcaagttcggagatgtgcagacagtgaaccccctagtggttaactggagagataataccgtgattagcagacccggccagtcccagtgtcccagattcaacacctgccctgaggtgtgctgggagggcgtgtacaacgacgccttcctgatcgatagaatcaactggatctctgccggcgtatttctggacagcaaccagaccgccgagaatcctgtgttcaccgtgttcaaggataacgaggtgctgtacagagcccagctggccagcgaggacacaaacgcccagaagaccatcacaaactgcttcctgctgaagaacaagatctggtgcatcagcctggtcgagatctacgacaccggcgacaacgtgatccggcctaagctgttcgctgtgaagatccctgagcagtgcacctgataaagctggagcctcggtggccttgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 9 - protein, 546 aa, synthetic construct
MAVILNKRYYSNLLLLILMISECSVGILHYEKLSKIGLVKGITRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSGYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 10 - RNA, 1638 nt, synthetic construct
  source 1..1638: mol_type = other RNA
atggccgtgatcctgaacaagcggtactactctaatctgctgctgctgatcctgatgatcagcgagtgcagcgtgggcattctccactacgagaagctgtccaaaatcggcctggtgaaaggcatcacaagaaagtacaagatcaaaagcaaccctctgaccaaggacatcgtgattaagatgatccctaatgtgagcaatatgagccagtgcaccggtagcgtgatggaaaactacaagaccagactgaacggcatcctgacccctatcaagggcgccctggaaatctataagaacaacacacatgacctggtgggagatgtgcggctggctggcgtgattatggccggcgtggccatcggaatcgccacagccgcccagatcaccgccggcgtcgccctgtacgaggccatgaaaaacgccgataatatcaacaagctgaagtctagcatcgagtctacaaacgaggccgtggtgaagctgcaggagacagcagagaagaccgtgtacgtgctgaccgccctgcaggactacattaacaccaatctggtgcctaccatcgacaagatctcatgtaagcagaccgagctgtctctggatctggccctgagcaaatatctgtctgatctgctgttcgtgttcggccctaacctgcaagaccctgtttccaattccatgacaatccaagccataagccaggccttcggcggcaattacgaaaccctgcttagaaccctgggctacgccacagaggacttcgacgacctgctggagagcgacagcatcaccggacagatcatctacgttgatctgtccggctactatatcatcgtgagagtgtacttccccatcctgactgagatccagcaggcttacatacaggagctgctgccagtgagcttcaacaacgacaattctgaatggatcagcatcgtgcccaacttcatcctggtgcggaacaccctcatcagcaacatcgagatcggattttgcctgatcaccaagcggagcgtgatctgcaaccaggattacgccacacctatgaccaacaatatgcgggaatgcctgacaggatctaccgagaagtgccccagagaactggtcgtgtccagccacgtgccaagattcgccctgtctaacggcgtgctgtttgccaactgcatctctgtgacctgtcagtgtcagacaaccggcagagccatcagccagagcggcgagcagaccctgctgatgatcgacaacaccacatgccctacagctgttctgggcaacgtgatcatttccctgggaaagtacctgggctctgtgaactataacagcgaaggcatcgcgattggacctcctgtgttcaccgacaaggtggacatcagcagccaaatcagtagcatgaaccagagcctgcagcagagcaaggactacattaaggaagctcagagactgctggacaccgtgaaccccagcctgatctctatgctgtctatgatcatcctgtacgtgctgagcatcgcctccctttgtatcggcctgatcaccttcatcagctttatcatcgtggaaaaaaagagaaacacctacagtagactggaagatagaagggtgcgccccacaagcagcggcgacctgtactacataggcacc

SEQ ID NO: 11 - RNA, 1931 nt, synthetic construct
  source 1..1931: mol_type = other RNA
aggccggcactcttctggtccccacagactcagagagaacccgccgccaccatggccgtgatcctgaacaagcggtactactctaatctgctgctgctgatcctgatgatcagcgagtgcagcgtgggcattctccactacgagaagctgtccaaaatcggcctggtgaaaggcatcacaagaaagtacaagatcaaaagcaaccctctgaccaaggacatcgtgattaagatgatccctaatgtgagcaatatgagccagtgcaccggtagcgtgatggaaaactacaagaccagactgaacggcatcctgacccctatcaagggcgccctggaaatctataagaacaacacacatgacctggtgggagatgtgcggctggctggcgtgattatggccggcgtggccatcggaatcgccacagccgcccagatcaccgccggcgtcgccctgtacgaggccatgaaaaacgccgataatatcaacaagctgaagtctagcatcgagtctacaaacgaggccgtggtgaagctgcaggagacagcagagaagaccgtgtacgtgctgaccgccctgcaggactacattaacaccaatctggtgcctaccatcgacaagatctcatgtaagcagaccgagctgtctctggatctggccctgagcaaatatctgtctgatctgctgttcgtgttcggccctaacctgcaagaccctgtttccaattccatgacaatccaagccataagccaggccttcggcggcaattacgaaaccctgcttagaaccctgggctacgccacagaggacttcgacgacctgctggagagcgacagcatcaccggacagatcatctacgttgatctgtccggctactatatcatcgtgagagtgtacttccccatcctgactgagatccagcaggcttacatacaggagctgctgccagtgagcttcaacaacgacaattctgaatggatcagcatcgtgcccaacttcatcctggtgcggaacaccctcatcagcaacatcgagatcggattttgcctgatcaccaagcggagcgtgatctgcaaccaggattacgccacacctatgaccaacaatatgcgggaatgcctgacaggatctaccgagaagtgccccagagaactggtcgtgtccagccacgtgccaagattcgccctgtctaacggcgtgctgtttgccaactgcatctctgtgacctgtcagtgtcagacaaccggcagagccatcagccagagcggcgagcagaccctgctgatgatcgacaacaccacatgccctacagctgttctgggcaacgtgatcatttccctgggaaagtacctgggctctgtgaactataacagcgaaggcatcgcgattggacctcctgtgttcaccgacaaggtggacatcagcagccaaatcagtagcatgaaccagagcctgcagcagagcaaggactacattaaggaagctcagagactgctggacaccgtgaaccccagcctgatctctatgctgtctatgatcatcctgtacgtgctgagcatcgcctccctttgtatcggcctgatcaccttcatcagctttatcatcgtggaaaaaaagagaaacacctacagtagactggaagatagaagggtgcgccccacaagcagcggcgacctgtactacataggcacctgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 4535 bp, synthetic construct
  source 1..4535: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagattaatacgactcactataaggccggcactcttctggtccccacagactcagagagaacccgccgccaccatggccgtgatcctgaacaagcggtactactctaatctgctgctgctgatcctgatgatcagcgagtgcagcgtgggcattctccactacgagaagctgtccaaaatcggcctggtgaaaggcatcacaagaaagtacaagatcaaaagcaaccctctgaccaaggacatcgtgattaagatgatccctaatgtgagcaatatgagccagtgcaccggtagcgtgatggaaaactacaagaccagactgaacggcatcctgacccctatcaagggcgccctggaaatctataagaacaacacacatgacctggtgggagatgtgcggctggctggcgtgattatggccggcgtggccatcggaatcgccacagccgcccagatcaccgccggcgtcgccctgtacgaggccatgaaaaacgccgataatatcaacaagctgaagtctagcatcgagtctacaaacgaggccgtggtgaagctgcaggagacagcagagaagaccgtgtacgtgctgaccgccctgcaggactacattaacaccaatctggtgcctaccatcgacaagatctcatgtaagcagaccgagctgtctctggatctggccctgagcaaatatctgtctgatctgctgttcgtgttcggccctaacctgcaagaccctgtttccaattccatgacaatccaagccataagccaggccttcggcggcaattacgaaaccctgcttagaaccctgggctacgccacagaggacttcgacgacctgctggagagcgacagcatcaccggacagatcatctacgttgatctgtccggctactatatcatcgtgagagtgtacttccccatcctgactgagatccagcaggcttacatacaggagctgctgccagtgagcttcaacaacgacaattctgaatggatcagcatcgtgcccaacttcatcctggtgcggaacaccctcatcagcaacatcgagatcggattttgcctgatcaccaagcggagcgtgatctgcaaccaggattacgccacacctatgaccaacaatatgcgggaatgcctgacaggatctaccgagaagtgccccagagaactggtcgtgtccagccacgtgccaagattcgccctgtctaacggcgtgctgtttgccaactgcatctctgtgacctgtcagtgtcagacaaccggcagagccatcagccagagcggcgagcagaccctgctgatgatcgacaacaccacatgccctacagctgttctgggcaacgtgatcatttccctgggaaagtacctgggctctgtgaactataacagcgaaggcatcgcgattggacctcctgtgttcaccgacaaggtggacatcagcagccaaatcagtagcatgaaccagagcctgcagcagagcaaggactacattaaggaagctcagagactgctggacaccgtgaaccccagcctgatctctatgctgtctatgatcatcctgtacgtgctgagcatcgcctccctttgtatcggcctgatcaccttcatcagctttatcatcgtggaaaaaaagagaaacacctacagtagactggaagatagaagggtgcgccccacaagcagcggcgacctgtactacataggcacctgactcgagtaagctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatgaagagcatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

(19) United States
(12) Patent Application Publication
(10) Pub. No.: US 2026/0240981 A1
(43) Pub. Date: Aug. 20, 2026

(54) ENGINEERED NIPAH VIRUS MRNA VACCINE

(71) Applicant: Vernagen, LLC, Tucker, GA (US)

(72) Inventors: Baek KIM, Atlanta, GA (US); Jack YOON, Buford, GA (US); David PAK, Suwanee, GA (US)

(73) Assignee: Vernagen, LLC, Tucker, GA (US)

(21) Appl. No.: 19/151,832

(22) PCT Filed: Feb. 9, 2024
(86) PCT No.: PCT/US2024/015123

§ 371 Date: Jul. 29, 2025

Related U.S. Application Data
(60) Provisional application No. 63/484,339, filed on Feb. 10, 2023.

(51) Int. Cl. A61K 39/155; A61K 39/00; A61P 31/14; C07K 14/005; C07K 14/78; C12N 7/00

(52) U.S. Cl. CPC A61K 39/155; A61P 31/14; C07K 14/005; C07K 14/78; C12N 7/00; A61K 2039/53; A61K 2039/575; C07K 2319/02; C12N 2760/18222; C12N 2760/18234; C12N 2830/50

(57) ABSTRACT

Provided herein are a Nipah virus vaccine composition including (i) a messenger ribonucleic acid (mRNA) including an open reading frame (ORF) encoding soluble Nipah virus glycoprotein (soluble NiV-G) fused with human collagen type I alpha 1 (COL1A1) signal peptide, (ii) a mRNA including an ORF encoding full-length Nipah virus glycoprotein (full-length NiV-G), (iii) a mRNA including an ORF encoding full-length Nipah virus fusion protein (full-length NiV-F), or (iv) the mRNA including the ORF encoding full-length NiV-G, and the mRNA including the ORF encoding full-length NiV-F, and a method of inducing immune response against Nipah virus by administering an effective amount of the Nipah virus vaccine composition to a subject in need thereof.

### CROSS REFERENCE TO RELATED APPLICATIONS

This application claims priority to U.S. Provisional Application No. 63/484,339 filed Feb. 10, 2023, the entire disclosure of which is incorporated herein by reference.

### INCORPORATION BY REFERENCE OF SEQUENCE LISTING

The content of the electronically submitted sequence listing, file name: F295207_sequence listing as filed; size: 37,709 bytes; and date of creation: Jan. 16, 2024, filed herewith, is incorporated herein by reference in its entirety.

### FIELD

Provided herein are a Nipah virus vaccine composition including (i) a messenger ribonucleic acid (mRNA) including an open reading frame (ORF) encoding soluble Nipah virus glycoprotein (soluble NiV-G) fused with human collagen type I alpha 1 (COL1A1) signal peptide, (ii) a mRNA including an ORF encoding full-length Nipah virus glycoprotein (full-length NiV-G), (iii) a mRNA including an ORF encoding full-length Nipah virus fusion protein (full-length NiV-F), or (iv) the mRNA including the ORF encoding full-length NiV-G, and the mRNA including the ORF encoding full-length NiV-F, and a method of inducing immune response against Nipah virus by administering an effective amount of the Nipah virus vaccine composition to a subject in need thereof.

### BACKGROUND

Nipah virus (NiV) is a zoonotic virus (it is transmitted from animals to humans) and can also be transmitted through contaminated food or directly between people. In infected people, it causes a range of illnesses from asymptomatic (subclinical) infection to acute respiratory illness and fatal encephalitis. At present, there is no approved Nipah virus mRNA vaccine, and there has been a need for Nipah virus mRNA vaccine.

### SUMMARY

The present disclosure provides a Nipah virus vaccine composition comprising a messenger ribonucleic acid (mRNA) comprising an open reading frame (ORF) encoding soluble Nipah virus glycoprotein (soluble NiV-G) fused with human collagen type I alpha 1 (COL1A1) signal peptide. In one embodiment, the soluble NiV-G fused with COL1A1 signal peptide has an amino acid sequence of SEQ ID NO: 1. In another embodiment, the ORF encoding soluble NiV-G fused with COL1A1 signal peptide has a nucleotide sequence of SEQ ID NO: 2. In some embodiment, the mRNA comprising the ORF encoding soluble NiV-G fused with COL1A1 signal peptide further comprises a 5′ untranslated region (UTR), a 3′ UTR, and a poly (A) tail so as to have the structure of 5′UTR-ORF encoding soluble NiV-G fused with COL1A1 signal peptide-3′UTR-poly (A) tail, and the ORF encoding soluble NiV-G fused with COL1A1 signal peptide has a nucleotide sequence of SEQ ID NO: 2. In one embodiment, the poly (A) tail has a length of 50-250 nucleotides. In some embodiment, the mRNA having the structure of 5′UTR-ORF encoding soluble NiV-G fused with COL1A1 signal peptide-3′UTR-poly (A) tail has a nucleotide sequence of SEQ ID NO: 3. In one embodiment, the mRNA having the structure of 5′UTR-ORF encoding soluble NiV-G fused with COL1A1 signal peptide-3′UTR-poly (A) tail has a nucleotide sequence having at least 80% identity to SEQ ID NO: 3. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

The present disclosure also provides a Nipah virus vaccine composition comprising a messenger ribonucleic acid (mRNA) comprising an open reading frame (ORF) encoding full-length Nipah virus glycoprotein (full-length NiV-G). In one embodiment, the full-length NiV-G has an amino acid sequence of SEQ ID NO: 5. In one embodiment, the ORF encoding full-length NiV-G has a nucleotide sequence of SEQ ID NO: 6. In one embodiment, the mRNA comprising the ORF encoding full-length NiV-G further comprises a 5′ untranslated region (UTR), a 3′ UTR, and a poly (A) tail so as to have the structure of 5′UTR-ORF encoding full-length NiV-G-3′UTR-poly (A) tail, and the ORF encoding full-length NiV-G has a nucleotide sequence of SEQ ID NO: 6. In one embodiment, the poly (A) tail has a length of 50-250 nucleotides. In one embodiment, the mRNA having the structure of 5′UTR-ORF encoding full-length NiV-G-3′UTR-poly (A) tail has a nucleotide sequence of SEQ ID NO: 7. In one embodiment, the mRNA having the structure of 5′UTR-ORF encoding full-length NiV-G-3′UTR-poly (A) tail has a nucleotide sequence having at least 80% identity to SEQ ID NO: 7. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

The present disclosure also provides a Nipah virus vaccine composition comprising a messenger ribonucleic acid (mRNA) comprising an open reading frame (ORF) encoding full-length Nipah virus fusion protein (full-length NiV-F). In one embodiment, the full-length NiV-F has an amino acid sequence of SEQ ID NO: 9. In one embodiment, the ORF encoding full-length NiV-F has a nucleotide sequence of SEQ ID NO: 10. In one embodiment, the mRNA comprising the ORF encoding full-length NiV-F further comprises a 5′ untranslated region (UTR), a 3′ UTR, and a poly (A) tail so as to have the structure of 5′UTR-ORF encoding full-length NiV-F-3′UTR-poly (A) tail, and the ORF encoding full-length NiV-F has a nucleotide sequence of SEQ ID NO: 10. In one embodiment, the poly (A) tail has a length of 50-250 nucleotides. In one embodiment, the mRNA having the structure of 5′UTR-ORF encoding full-length NiV-F-3′UTR-poly (A) tail has a nucleotide sequence of SEQ ID NO: 11. In one embodiment, the mRNA having the structure of 5′UTR-ORF encoding full-length NiV-F-3′UTR-poly (A) tail has a nucleotide sequence having at least 80% identity to SEQ ID NO: 11. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

The present disclosure also provides a Nipah virus vaccine composition comprising a messenger ribonucleic acid (mRNA) comprising an open reading frame (ORF) encoding full-length Nipah virus glycoprotein (full-length NiV-G), and a mRNA comprising an ORF encoding full-length Nipah virus fusion protein (full-length NiV-F). In one embodiment, the full-length NiV-G has an amino acid sequence of SEQ ID NO: 5, and the full-length NiV-F has an amino acid sequence of SEQ ID NO: 9. In one embodiment, the ORF encoding full-length NiV-G has a nucleotide sequence of SEQ ID NO: 6, and the ORF encoding full-length NiV-F has a nucleotide sequence of SEQ ID NO: 10. In one embodiment, the mRNA comprising the ORF encoding full-length NiV-G further comprises a 5′ untranslated region (UTR), a 3′ UTR, and a poly (A) tail so as to have the structure of 5′UTR-ORF encoding full-length NiV-G-3′UTR-poly (A) tail, and the ORF encoding full-length NiV-G has a nucleotide sequence of SEQ ID NO: 6, and the mRNA comprising the ORF encoding full-length NiV-F further comprises a 5′ UTR, a 3′ UTR, and a poly (A) tail so as to have the structure of 5′UTR-ORF encoding full-length NiV-F-3′UTR-poly (A) tail, and the ORF encoding full-length NiV-F has a nucleotide sequence of SEQ ID NO: 10. In one embodiment, the poly (A) tail has a length of 50-250 nucleotides. In one embodiment, the mRNA having the structure of 5′UTR-ORF encoding full-length NiV-G-3′UTR-poly (A) tail has a nucleotide sequence of SEQ ID NO: 7, and the mRNA having the structure of 5′UTR-ORF encoding full-length NiV-F-3′UTR-poly (A) tail has a nucleotide sequence of SEQ ID NO: 11. In one embodiment, the mRNA having the structure of 5′UTR-ORF encoding full-length NiV-G-3′UTR-poly (A) tail has a nucleotide sequence having at least 80% identity to SEQ ID NO: 7, and the mRNA having the structure of 5′UTR-ORF encoding full-length NiV-F-3′UTR-poly (A) tail has a nucleotide sequence having at least 80% identity to SEQ ID NO: 11. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

The present disclosure also provides a method of inducing immune response against Nipah virus comprising administering an effective amount of the Nipah virus vaccine composition according to the present disclosure to a subject in need thereof.

### BRIEF DESCRIPTION OF THE DRAWINGS

FIG. 1 shows in vitro transcription for NiV mRNAs.

FIGS. 2A and 2B show NiV-G and NiV-F protein expression in mRNA-transfected 293FT cells.

FIG. 3A describes a mouse immunization schedule using the Nipah virus vaccine compositions of the present disclosure.

FIG. 3B shows the data as to mouse anti-NiV-GP titer.

FIG. 3C shows the data as to NiV neutralization.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this disclosure is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this disclosure belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc., without the exclusion of the presence of additional feature(s), element(s), method step(s), etc. Conversely, the term “consisting of” and linguistic variations thereof, denotes the presence of recited feature(s), element(s), method step(s), etc., and excludes any unrecited feature(s), element(s), method step(s), etc., except for ordinarily-associated impurities. The phrase “consisting essentially of” denotes the recited feature(s), element(s), method step(s), etc., and any additional feature(s), element(s), method step(s), etc., that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “Nipah virus vaccine composition” refers to a substance used to stimulate the production of antibodies and provide immunity against Nipah virus.

As used herein, the term “messenger ribonucleic acid (mRNA)” refers to a single-stranded molecule of RNA that corresponds to the genetic sequence of a gene, and is read by a ribosome in the process of synthesizing a protein.

As used herein, the term “fused with” refers to a gene or gene product which has the characteristics of that gene or gene product when isolated from a naturally occurring source.

The term “Nipah virus glycoprotein (soluble NiV-G) fused with human collagen type I alpha 1 (COL1A1) signal peptide” refers to a recombinant fusion protein created through genetic engineering of a fusion gene. For instance, this may involve removing the stop codon from a cDNA sequence coding for soluble NiV-G, then appending the cDNA sequence of COL1A1 signal peptide in frame through ligation or overlap extension PCR.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “open reading frame (ORF)” refers to a nucleotide sequence between the start and stop codons.

As used herein, the term “an open reading frame (ORF) encoding” refers to the nucleotide coding sequence which encodes a polypeptide. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

As used herein, the term “T7 promoter” refers to a promoter derived from a bacteriophage T7.

As used herein, the term “5′ untranslated region (UTR)” refers to a region of an mRNA that is directly upstream (i.e., 5′) from the start codon (the first codon of an mRNA transcript translated by a ribosome) that does not encode a polypeptide.

As used herein, the term “3′ untranslated region (UTR)” refers to a region of an mRNA that is directly downstream (i.e., 3′) from the stop codon (i.e., the codon of an mRNA transcript that signals a termination of translation) that does not encode a polypeptide.

As used herein, the term “poly (A) tail” refers to a long stretch of adenine nucleotides added to the “tail” or 3′ end of the mRNA.

As used herein, the term “pharmaceutically acceptable carrier” refers to any substance or vehicle suitable for delivering a mRNA vaccine to a suitable in vivo or ex vivo site. Such a carrier can include, but is not limited to, an adjuvant, an excipient, a lipid particle, etc.

As used herein, the term “lipid nanoparticle” refers to a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

As used herein, the term “inducing immune response against Nipah virus” refers to providing protective immunity and/or vaccinating a subject against Nipah virus for prophylactic purposes, as well as causing a desired immune response or effect in a subject in need thereof against Nipah virus, for therapeutic purposes. As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all.

An “effective amount” of the Nipah virus vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the Nipah virus vaccine (e.g., mRNA) provides an induced or boosted immune response as a function of antigen production in the cell, preferably more efficient than a composition containing a corresponding unmodified polynucleotide encoding the same antigen or a peptide antigen.

Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

As used herein, the term “X % identity to SEQ ID NO: Y” or “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “nucleotide sequence having at least X % identity to SEQ ID NO: Y and encodes Z protein” means that the nucleotide sequence meets the two different requirements of having at least X % identity to SEQ ID NO: Y and encoding Z protein.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The terms “subject,” “patient,” “individual,” and the like are used interchangeably herein, and refer to any animal, any mammalian subject, or cells thereof whether in vitro or in situ, amenable to the methods described herein. In certain non-limiting embodiments, the patient, subject or individual is a human.

### DETAILED DESCRIPTION

### 1. The Nipah Virus Vaccine Compositions

(1) Nipah Virus Vaccine Composition (1): A Nipah Virus Vaccine Composition Comprising a mRNA Comprising an ORF Encoding Soluble Nipah Virus Glycoprotein (Soluble NiV-G) Fused with Human Collagen Type I Alpha 1 (COL1A1) Signal Peptide

The present disclosure provides a Nipah virus vaccine composition comprising a messenger ribonucleic acid (mRNA) comprising an open reading frame (ORF) encoding soluble Nipah virus glycoprotein (soluble NiV-G) fused with human collagen type I alpha 1 (COL1A1) signal peptide. In one embodiment, the soluble NiV-G fused with COL1A1 signal peptide has an amino acid sequence of SEQ ID NO: 1 (or an amino acid sequence having at least 80%, 85%, 90%, 95%, 96%, 97%, 98% or 99% identity to SEQ ID NO: 1). In another embodiment, the ORF encoding soluble NiV-G fused with COL1A1 signal peptide has a nucleotide sequence of SEQ ID NO: 2 (or a nucleotide sequence having at least 80%, 85%, 90%, 95%, 96%, 97%, 98% or 99% identity to SEQ ID NO: 2). In some embodiment, the mRNA comprising the ORF encoding soluble NiV-G fused with COL1A1 signal peptide further comprises a 5′ untranslated region (UTR), a 3′ UTR, and a poly (A) tail so as to have the structure of 5′UTR-ORF encoding soluble NiV-G fused with COL1A1 signal peptide-3′UTR-poly (A) tail, and the ORF encoding soluble NiV-G fused with COL1A1 signal peptide has a nucleotide sequence of SEQ ID NO: 2 (or a nucleotide sequence having at least 80%, 85%, 90%, 95%, 96%, 97%, 98% or 99% identity to SEQ ID NO: 2). In one embodiment, the poly (A) tail has a length of 50-250 nucleotides. In some embodiment, the mRNA having the structure of 5′UTR-ORF encoding soluble NiV-G fused with COL1A1 signal peptide-3′UTR-poly (A) tail has a nucleotide sequence of SEQ ID NO: 3 (or a nucleotide sequence having at least 80%, 85%, 90%, 95%, 96%, 97%, 98% or 99% identity to SEQ ID NO: 3). In one embodiment, the mRNA having the structure of 5′UTR-ORF encoding soluble NiV-G fused with COL1A1 signal peptide-3′UTR-poly (A) tail has a nucleotide sequence having at least 80% identity to SEQ ID NO: 3. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

(2) Nipah Virus Vaccine Composition (2): A Nipah Virus Vaccine Composition Comprising a mRNA Comprising an ORF Encoding Full-Length Nipah Virus Glycoprotein (Full-Length NiV-G)

The present disclosure also provides a Nipah virus vaccine composition comprising a messenger ribonucleic acid (mRNA) comprising an open reading frame (ORF) encoding full-length Nipah virus glycoprotein (full-length NiV-G). In one embodiment, the full-length NiV-G has an amino acid sequence of SEQ ID NO: 5 (or an amino acid sequence having at least 80%, 85%, 90%, 95%, 96%, 97%, 98% or 99% identity to SEQ ID NO: 5). In one embodiment, the ORF encoding full-length NiV-G has a nucleotide sequence of SEQ ID NO: 6 (or a nucleotide sequence having at least 80%, 85%, 90%, 95%, 96%, 97%, 98% or 99% identity to SEQ ID NO: 6). In one embodiment, the mRNA comprising the ORF encoding full-length NiV-G further comprises a 5′ untranslated region (UTR), a 3′ UTR, and a poly (A) tail so as to have the structure of 5′UTR-ORF encoding full-length NiV-G-3′UTR-poly (A) tail, and the ORF encoding full-length NiV-G has a nucleotide sequence of SEQ ID NO: 6 (or a nucleotide sequence having at least 80%, 85%, 90%, 95%, 96%, 97%, 98% or 99% identity to SEQ ID NO: 6). In one embodiment, the poly (A) tail has a length of 50-250 nucleotides. In one embodiment, the mRNA having the structure of 5′UTR-ORF encoding full-length NiV-G-3′UTR-poly (A) tail has a nucleotide sequence of SEQ ID NO: 7 (or a nucleotide sequence having at least 80%, 85%, 90%, 95%, 96%, 97%, 98% or 99% identity to SEQ ID NO: 7). In one embodiment, the mRNA having the structure of 5′UTR-ORF encoding full-length NiV-G-3′UTR-poly (A) tail has a nucleotide sequence having at least 80% identity to SEQ ID NO: 7. In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

(3) Nipah Virus Vaccine Composition (3): A Nipah Virus Vaccine Composition Comprising a mRNA Comprising an ORF Encoding Full-Length Nipah Virus Fusion Protein (Full-Length NiV-F)

The present disclosure also provides a Nipah virus vaccine composition comprising a messenger ribonucleic acid (mRNA) comprising an open reading frame (ORF) encoding full-length Nipah virus fusion protein (full-length NiV-F). In one embodiment, the full-length NiV-F has an amino acid sequence of SEQ ID NO: 9 (or an amino acid sequence having at least 80%, 85%, 90%, 95%, 96%, 97%, 98% or 99% identity to SEQ ID NO: 9). In one embodiment, the ORF encoding full-length NiV-F has a nucleotide sequence of SEQ ID NO: 10 (or a nucleotide sequence having at least 80%, 85%, 90%, 95%, 96%, 97%, 98% or 99% identity to SEQ ID NO: 10). In one embodiment, the mRNA comprising the ORF encoding full-length NiV-F further comprises a 5′ untranslated region (UTR), a 3′ UTR, and a poly (A) tail so as to have the structure of 5′UTR-ORF encoding full-length NiV-F-3′UTR-poly (A) tail, and the ORF encoding full-length NiV-F has a nucleotide sequence of SEQ ID NO: 10 (or a nucleotide sequence having at least 80%, 85%, 90%, 95%, 96%, 97%, 98% or 99% identity to SEQ ID NO: 10). In one embodiment, the poly (A) tail has a length of 50-250 nucleotides. In one embodiment, the mRNA having the structure of 5′UTR-ORF encoding full-length NiV-F-3′UTR-poly (A) tail has a nucleotide sequence of SEQ ID NO: 11 (or a nucleotide sequence having at least 80%, 85%, 90%, 95%, 96%, 97%, 98% or 99% identity to SEQ ID NO: 11). In one embodiment, the mRNA having the structure of 5′UTR-ORF encoding full-length NiV-F-3′UTR-poly (A) tail has a nucleotide sequence having at least 80% identity to SEQ ID NO: 11 (or a nucleotide sequence having at least 80%, 85%, 90%, 95%, 96%, 97%, 98% or 99% identity to SEQ ID NO: 11). In one embodiment, the Nipah virus composition of the present disclosure further comprises a pharmaceutically acceptable carrier. In one embodiment, the pharmaceutically acceptable carrier is a lipid nanoparticle encapsulating the mRNA therein.

(4) Nipah Virus Vaccine Composition (4): A Nipah Virus Vaccine Composition Comprising a mRNA Comprising an ORF Encoding Full-Length Nipah Virus Glycoprotein (Full-Length NiV-G) and an ORF Encoding Full-Length Nipah Virus Fusion Protein (Full-Length NiV-F)

The present disclosure also provides a Nipah virus vaccine composition comprising a messenger ribonucleic acid (mRNA) comprising an open reading frame (ORF) encoding full-length Nipah virus glycoprotein (full-length NiV-G), and a mRNA comprising an ORF encoding full-length Nipah virus fusion protein (full-length NiV-F). In one embodiment, the full-length NiV-G has an amino acid sequence of SEQ ID NO: 5, and the full-length NiV-F has an amino acid sequence of SEQ ID NO: 9. In one embodiment, the ORF encoding full-length NiV-G has a nucleotide sequence of SEQ ID NO: 6, and the ORF encoding full-length NiV-F has a nucleotide sequence of SEQ ID NO: 10. In one embodiment, the mRNA comprising the ORF encoding full-length NiV-G further comprises a 5′ untranslated region (UTR), a 3′ UTR, and a poly (A) tail so as to have the structure of 5′UTR-ORF encoding full-length NiV-G-3′UTR-poly (A) tail, and the ORF encoding full-length NiV-G has a nucleotide sequence of SEQ ID NO: 6, and the mRNA comprising the ORF encoding full-length NiV-F further comprises a 5′ UTR, a 3′ UTR, and a poly (A) tail so as to have the structure of 5′UTR-ORF encoding full-length NiV-F-3′UTR-poly (A) tail, and the ORF encoding full-length NiV-F has a nucleotide sequence of SEQ ID NO: 10. In one embodiment, the poly (A) tail has a length of 50-250 nucleotides. In one embodiment, the mRNA having the structure of 5′UTR-ORF encoding full-length NiV-G-3′UTR-poly (A) tail has a nucleotide sequence of SEQ ID NO: 7, and the mRNA having the structure of 5′UTR-ORF encoding full-length NiV-F-3′UTR-poly (A) tail has a nucleotide sequence of SEQ ID NO: 11. In one embodiment, the mRNA having the structure of 5′UTR-ORF encoding full-length NiV-G-3′UTR-poly (A) tail has a nucleotide sequence having at least 80% identity to SEQ ID NO: 7, and the mRNA having the structure of 5′UTR-ORF encoding full-length NiV-F-3′UTR-poly (A) tail has a nucleotide sequence having at least 80% identity to SEQ ID NO: 11. In one embodiment, the Nipah virus composition of the present disclosure further comprises a pharmaceutically acceptable carrier. In one embodiment, the mRNA encoding the full-length NiV-G and the mRNA encoding the full-length Ni-F are included in the composition at a ratio of about 1:1. In one embodiment, the pharmaceutically acceptable carrier is a lipid nanoparticle encapsulating the mRNA therein.

In the above Nipah Virus vaccine compositions (1) to (4), the poly (A) tail may have a length of 50-250 nucleotides. In another embodiment, the poly (A) tail has a length of 100-200 nucleotides. In another embodiment, the poly (A) tail has a length of 110-150 nucleotides. In another embodiment, the poly (A) tail has a length of 115-125 nucleotides. In another embodiment, the poly (A) tail has a length of 116-124 nucleotides. In another embodiment, the poly (A) tail has a length of 117-123 nucleotides. In another embodiment, the poly (A) tail has a length of 118-122 nucleotides. In another embodiment, the poly (A) tail has a length of 119-122 nucleotides. In another embodiment, the poly (A) tail has a length of 115 nucleotides. In another embodiment, the poly (A) tail has a length of 116 nucleotides. In another embodiment, the poly (A) tail has a length of 117 nucleotides. In another embodiment, the poly (A) tail has a length of 118 nucleotides. In another embodiment, the poly (A) tail has a length of 119 nucleotides. In another embodiment, the poly (A) tail has a length of 120 nucleotides. In another embodiment, the poly (A) tail has a length of 121 nucleotides. In another embodiment, the poly (A) tail has a length of 122 nucleotides. In another embodiment, the poly (A) tail has a length of 123 nucleotides. In another embodiment, the poly (A) tail has a length of 124 nucleotides. In another embodiment, the poly (A) tail has a length of 125 nucleotides.

In the above Nipah Virus vaccine compositions (1) to (4), the mRNA of the present disclosure may comprise at least one chemical modification selected from the group consisting of pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methylpseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methylpseudouridine, 4-thio-pseudouridine, 5-aza-uridine, dihydropseudouridine, 5-methoxyuridine and 2′-O-methyl uridine. In another embodiment, the chemical modification is in the 5-position of the uracil. In another embodiment, the chemical modification is a N1-methylpseudouridine. In another embodiments, the chemical modification is a N1-ethylpseudouridine.

In the above Nipah Virus vaccine compositions (1) to (4), the Nipah virus vaccine composition may further comprise a pharmaceutically acceptable carrier. In another embodiment, the pharmaceutically acceptable carrier may include any substance or vehicle suitable for delivering a mRNA vaccine to a suitable in vivo or ex vivo site. Such a carrier can include, but is not limited to, an adjuvant, an excipient, a lipid particle, etc. The lipid nanoparticle may be a particle having at least one dimension on the order of nanometers (e.g., 1-1,000 nm). In some embodiments, lipid nanoparticles are included in a formulation that can be used to deliver a mRNA vaccine to a target site of interest (e.g., cell, tissue, organ, tumor, and the like). In some embodiments, the mRNA vaccine, may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells, e.g., an adverse immune response. In some embodiments, the lipid nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

In one embodiment, the lipid nanoparticle comprises (i) at least one lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), (ii) a neutral lipid selected from DSPC, DPPC, POPC, DOPE and SM, (iii) a sterol, e.g., cholesterol, and (iv) a PEG-lipid, e.g., PEG-DMG or PEG-cDMA, in a molar ratio of about 20-60% cationic lipid:5-25% neutral lipid:25-55% sterol; 0.5-15% PEG-lipid.

In one embodiment, the lipid nanoparticle includes from about 25% to about 75% on a molar basis of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., from about 35 to about 65%, from about 45 to about 65%, about 60%, about 57.5%, about 50% or about 40% on a molar basis.

In one embodiment, the lipid nanoparticle includes from about 0.5% to about 15% on a molar basis of the neutral lipid e.g., from about 3 to about 12%, from about 5 to about 10% or about 15%, about 10%, or about 7.5% on a molar basis. Examples of neutral lipids include, but are not limited to, DSPC, POPC, DPPC, DOPE and SM. In some embodiments, the formulation includes from about 5% to about 50% on a molar basis of the sterol (e.g., about 15 to about 45%, about 20 to about 40%, about 40%, about 38.5%, about 35%, or about 31% on a molar basis. An exemplary sterol is cholesterol. In some embodiments, the formulation includes from about 0.5% to about 20% on a molar basis of the PEG or PEG-modified lipid (e.g., about 0.5 to about 10%, about 0.5 to about 5%, about 1.5%, about 0.5%, about 1.5%, about 3.5%, or about 5% on a molar basis. In some embodiments, the PEG or PEG modified lipid comprises a PEG molecule of an average molecular weight of 2,000 Da. In other embodiments, the PEG or PEG modified lipid comprises a PEG molecule of an average molecular weight of less than 2,000, for example around 1,500 Da, around 1,000 Da, or around 500 Da. Examples of PEG-modified lipids include, but are not limited to, PEG-distearoyl glycerol (PEG-DMG) (also referred herein as PEG-C14 or C14-PEG), and PEG-cDMA.

In one embodiment, the lipid nanoparticle includes 25-75% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In one embodiment, the lipid nanoparticle include 35-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In one embodiment, the lipid nanoparticle includes 45-65% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In one embodiment, the lipid nanoparticle includes about 60% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 7.5% of the neutral lipid, about 31% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 38.5% of the sterol, and about 1.5% of the PEG or PEG-modified lipid on a molar basis.

In one embodiment, the lipid nanoparticle includes about 50% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 10% of the neutral lipid, about 35% of the sterol, about 4.5% or about 5% of the PEG or PEG-modified lipid, and about 0.5% of the targeting lipid on a molar basis.

In one embodiment, the lipid nanoparticle includes about 40% of a cationic lipid selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), about 15% of the neutral lipid, about 40% of the sterol, and about 5% of the PEG or PEG-modified lipid on a molar basis.

In one embodiment, the Nipah virus vaccine composition of the present disclosure may be delivered, localized and/or concentrated in a specific location using the delivery methods described as follows. As a non-limiting example, a subject may be administered an empty polymeric particle prior to, simultaneously with or after delivering the Nipah virus vaccine composition of the present disclosure to the subject. The empty polymeric particle undergoes a change in volume once in contact with the subject and becomes lodged, embedded, immobilized or entrapped at a specific location in the subject.

In another embodiment, the Nipah virus vaccine composition of the present disclosure may be formulated in an active substance release system. For instance, the active substance release system may comprise at least one nanoparticle bonded to an oligonucleotide inhibitor strand which is hybridized with a catalytically active nucleic acid and a compound bonded to at least one substrate molecule bonded to a therapeutically active substance (e.g., polynucleotides described herein), where the therapeutically active substance is released by the cleavage of the substrate molecule by the catalytically active nucleic acid.

In another embodiment, the Nipah virus vaccine composition of the present disclosure may be formulated in a nanoparticle comprising an inner core comprising a non-cellular material and an outer surface comprising a cellular membrane. The cellular membrane may be derived from a cell or a membrane derived from a virus.

In another embodiment, the Nipah virus vaccine composition of the present disclosure may be formulated in porous nanoparticle-supported lipid bilayers (protocells).

In another embodiment, the Nipah virus vaccine composition of the present disclosure may be formulated in polymeric nanoparticles which have a high glass transition temperature.

In another embodiment, the Nipah virus vaccine composition of the present disclosure may be formulated in nanoparticles used in imaging. As a non-limiting example, the liposome may comprise gadolinium(III)2-{4,7-bis-carboxymethyl-10-[(N,N-distearylamidomethyl-N′-amido-methyl]-1,4,7,10-tetra-azacyclododec-1-yl}-acetic acid and a neutral, fully saturated phospholipid component.

The nanoparticles of the present disclosure may further include nutrients such as, but not limited to, those which deficiencies can lead to health hazards from anemia to neural tube defects. As a non-limiting example, the nutrient may be iron in the form of ferrous, ferric salts or elemental iron, iodine, folic acid, vitamins or micronutrients.

In another embodiment, the Nipah virus vaccine composition of the present disclosure may be formulated in a swellable nanoparticle.

In another embodiment, the Nipah virus vaccine composition of the present disclosure may be formulated in polyanhydride nanoparticles.

The nanoparticles and microparticles of the present disclosure may be geometrically engineered to modulate macrophage and/or the immune response. In some embodiments, the geometrically engineered particles may have varied shapes, sizes and/or surface charges in order to incorporated the polynucleotides of the present disclosure for targeted delivery such as, but not limited to, pulmonary delivery. Other physical features the geometrically engineering particles may have include, but are not limited to, fenestrations, angled arms, asymmetry and surface roughness, charge which can alter the interactions with cells and tissues.

In another embodiment, the nanoparticles of the present disclosure may be water soluble nanoparticles. The nanoparticles may be inorganic nanoparticles which have a compact and zwitterionic ligand in order to exhibit good water solubility. The nanoparticles may also have small hydrodynamic diameters (HD), stability with respect to time, pH, and salinity and a low level of non-specific protein binding.

In some embodiments, the nanoparticles of the present disclosure are stealth nanoparticles or target-specific stealth nanoparticles. In some embodiments, the stealth or target-specific stealth nanoparticles may comprise a polymeric matrix. The polymeric matrix may comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates or combinations thereof.

In one embodiment, the nanoparticle of the present disclosure may be a nanoparticle-nucleic acid hybrid structure having a high density nucleic acid layer. The nanoparticle of the present disclosure may comprise a nucleic acid such as, but not limited to, polynucleotides described herein and/or known in the art.

In one embodiment, at least one of the nanoparticles of the present disclosure may be embedded in in the core a nanostructure or coated with a low density porous 3-D structure or coating which is capable of carrying or associating with at least one payload within or on the surface of the nanostructure.

In one embodiment, the pharmaceutically acceptable carrier is a lipid nanoparticle encapsulating the mRNAs of the present disclosure therein. In another embodiment, the lipid nanoparticle comprises a first lipid nanoparticle encapsulating the mRNA encoding soluble NiV-G fused with COL1A1 signal peptide, a second lipid nanoparticle encapsulating the mRNA encoding full-length NiV-G, a third lipid nanoparticle encapsulating the mRNA encoding full-length NiV-F, and a fourth lipid nanoparticle encapsulating the mRNA encoding full-length NiV-G and the mRNA encoding full-length NiV-F therein.

### 2. The Method of Inducing Immune Response Against Nipah Virus

The present disclosure also provides a method of inducing immune response against Nipah virus comprising administering an effective amount of the Nipah virus vaccine composition of the present disclosure a subject in need thereof. In one embodiment, the effective amount of the Nipah virus vaccine composition (e.g. mRNA) is provided based, at least in part, on the target tissue, target cell type, means of administration, physical characteristics of the polynucleotide (e.g., size, and extent of modified nucleosides) and other components of the vaccine, and other determinants. In general, an effective amount of the Nipah virus vaccine (e.g., mRNA) provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA, e.g., mRNA, vaccine), increased protein translation from the polynucleotide, decreased nucleic acid degradation (as demonstrated, for example, by increased duration of protein translation from a modified polynucleotide), or altered antigen specific immune response of the host cell.

Administration of an effective amount (immunogenically effective amount) of the Nipah virus vaccine compositions (e.g., Nipah virus vaccine compositions (1) to (4)) is typically intramuscular or subcutaneous. Thus, the Nipah virus vaccine composition is typically formulated for intramuscular or subcutaneous injection, and for the purposes of the invention formulated without adjuvants, preferably without any adjuvant. However other modes of administration, such as intravenous, cutaneous, intradermal or nasal can be envisaged as well. For intravenous, cutaneous or subcutaneous injection, the adenovirus vector will be in the form of a parenterally acceptable aqueous solution which is pyrogen-free and has suitable pH, isotonicity and stability. Likewise, the isolated envelope polypeptide will be in the form of a parenterally acceptable solution having a suitable pH, isotonicity, and stability. Those of ordinary skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In a particular embodiment, an effective amount (immunogenically effective amount) of the Nipah virus vaccine composition (e.g., Nipah virus vaccine compositions (1) to (4)) is administered via intramuscular administration. Intramuscular administration can be achieved by using a needle to inject a suspension of the adenovirus vectors and/or envelope polypeptides. An alternative is the use of a needleless injection device to administer the composition (using, e.g., Biojector™) or a freeze-dried powder containing the vaccine.

In one embodiment, the priming immunization and/or the boosting administration, preferably both the priming and boosting administration, further comprise administering one or more adenovirus vectors that encode one or more further Nipah virus antigens.

The timing for administering priming and boosting immunizations is not particularly limited. For example, a vaccine composition can be administered for priming immunization, and re-administered prior to administration of a vaccine composition for boosting immunization. Further administrations of a vaccine composition for further boosting immunizations are also contemplated. In certain embodiments, a booster vaccine is first administered about 1-12 weeks, e.g., 1, 2, 3, 4, 5, 6, 7, 8, 9, 10, 11, or 12 weeks after a primer vaccine is initially administered. In other embodiments, a booster vaccine is first administered about 12-52 weeks, e.g., about 12, 14, 16, 18, 20, 22, 24, 26, 28, 30, 32, 34, 36, 38, 40, 42, 44, 46, 48, 50, or 52 weeks after a primer vaccine is initially administered. One of ordinary skill in the art will be able to vary the exact timing of the priming and boosting vaccines, frequency of administration thereof, dosage thereof, etc., based upon the teachings herein and general knowledge in the art.

In one embodiment, the Nipah virus vaccine composition may comprise the first and second mRNAs described herein, formulated in a lipid nanoparticle comprising MC3, Cholesterol, DSPC and PEG2000-DMG, the buffer trisodium citrate, sucrose and water for injection. As a non-limiting example, the composition may comprise 2.0 mg/mL of drug substance (e.g., Nipah virus vaccine compositions (1) to (4)), 21.8 mg/mL of MC3, 10.1 mg/mL of cholesterol, 5.4 mg/mL of DSPC, 2.7 mg/mL of PEG2000-DMG, 5.16 mg/mL of trisodium citrate, 71 mg/mL of sucrose and 1.0 mL of water for injection.

In one embodiment, a method of inducing immune response against Nipah virus comprises administering an effective amount of the Nipah virus vaccine composition (1) of the present disclosure to a subject in need thereof. In the Nipah virus vaccine composition (1), the mRNA having the structure of 5′UTR-ORF encoding soluble NiV-G fused with COL1A1 signal peptide −3′UTR-poly (A) tail may have a nucleotide sequence of SEQ ID NO: 3 (or a nucleotide sequence having at least 80%, 85%, 90%, 95%, 96%, 97%, 98%, 99% or 99% identity to SEQ ID NO: 3).

In another embodiment, a method of inducing immune response against Nipah virus comprises administering an effective amount of the Nipah virus vaccine composition (2) of the present disclosure to a subject in need thereof. In the Nipah virus vaccine composition (2), the mRNA having the structure of 5′UTR-ORF encoding full-length NiV-G-3′UTR-poly (A) tail may have a nucleotide sequence of SEQ ID NO: 7 (or a nucleotide sequence having at least 80%, 85%, 90%, 95%, 96%, 97%, 98%, 99% or 99% identity to SEQ ID NO: 7).

In another embodiment, a method of inducing immune response against Nipah virus comprises administering an effective amount of the Nipah virus vaccine composition (3) of the present disclosure to a subject in need thereof. In the Nipah virus vaccine composition (3), the mRNA having the structure of 5′UTR-ORF encoding full-length NiV-F-3′UTR-poly (A) tail may have a nucleotide sequence of SEQ ID NO: 11 (or a nucleotide sequence having at least 80%, 85%, 90%, 95%, 96%, 97%, 98%, 99% or 99% identity to SEQ ID NO: 11).

In another embodiment, a method of inducing immune response against Nipah virus comprises administering an effective amount of the Nipah virus vaccine composition (4) of the present disclosure to a subject in need thereof. In the Nipah virus vaccine composition (4), the mRNA having the structure of 5′UTR-ORF encoding full-length NiV-G-3′UTR-poly (A) tail may have a nucleotide sequence of SEQ ID NO: 7 (or a nucleotide sequence having at least 80%, 85%, 90%, 95%, 96%, 97%, 98%, 99% or 99% identity to SEQ ID NO: 7), and the mRNA having the structure of 5′UTR-ORF encoding full-length NiV-F-3′UTR-poly (A) tail may have a nucleotide sequence of SEQ ID NO: 11 (or a nucleotide sequence having at least 80%, 85%, 90%, 95%, 96%, 97%, 98%, 99% or 99% identity to SEQ ID NO: 11).

3. Sequence Information
1) Protein sequence of the soluble NiV-G fused with COL1A1
signal peptide (COL1A1 signal peptide sequence is underlined)
SEQ ID NO: 1
MFSFVDLRLLLLLAATALLTHGQNYTRSTDNQAMIKDALQSIQQQIKGLADKIGTEIGPK
VSLIDTSSTITIPANIGLLGSKISQSTASINENVNEKCKFTLPPLKIHECNISCPNPLPFREYK
PQTEGVSNLVGLPNNICLQKTSNQILKPKLISYTLPVVGQSGTCITDPLLAMDEGYFAYS
HLEKIGSCSRGVSKQRIIGVGEVLDRGDEVPSLFMTNVWTPSNPNTVYHCSAVYNSEFY
YVLCAVSVVGDPILNSTYWSGSLMMTRLAVKPKNNGESYNQHQFALRNIEKGMYDKV
MPYGPSGIKQGDTLYFPAVGFLVRTEFKYNDSNCPVAKCQYSKPENCRLSMGIRPNSHY
ILRSGLLKYNLSDEENSKIIFIEISDQRLSIGSPSKIYDSLGQPVFYQASFSWDTMIKFGDVQ
TVNPLVVNWRDNTVISRPGQSQCPRFNTCPEVCWEGVYNDAFLIDRINWISAGVFLDSN
QTAENPVFTVFKDNEVLYRAQLASEDTNAQKTITNCFLLKNKIWCISLVEIYDTGDNVIR
PKLFAVKIPEQCT
2) Soluble NiV-G fused with COL1A1 signal peptide mRNA sequence (ORF)
SEQ ID NO: 2
AUGUUCUCUUUCGUGGACCUGCGCCUGCUGCUGCUGCUCGCUGCCACCGCCCUGC
UGACACACGGCCAGAACUACACCCGGAGCACCGACAACCAGGCUAUGAUCAAAGA
CGCCCUGCAGAGCAUCCAACAGCAGAUCAAGGGCCUGGCCGACAAGAUCGGCACA
GAAAUCGGACCAAAGGUGUCCCUGAUCGACACGAGUUCUACAAUCACUAUCCCUG
CCAACAUCGGCCUGCUGGGCAGCAAAAUCUCUCAGAGCACAGCCAGCAUCAACGA
GAACGUGAACGAGAAAUGCAAGUUCACCCUGCCACCACUGAAGAUCCACGAAUGC
AACAUCAGCUGCCCUAAUCCUCUGCCCUUCAGAGAGUACAAGCCUCAGACCGAGG
GUGUGUCUAACCUGGUGGGCCUGCCGAACAACAUCUGCCUGCAAAAAACCAGCAA
CCAGAUCCUGAAACCUAAGCUGAUCAGCUACACACUGCCUGUGGUGGGCCAGAGC
GGCACCUGUAUCACAGAUCCUCUGCUGGCCAUGGAUGAAGGCUACUUCGCCUACA
GCCAUCUGGAAAAGAUCGGGAGCUGUAGCCGGGGCGUCUCCAAACAGAGAAUCA
UCGGCGUGGGCGAAGUGCUGGACAGAGGCGAUGAGGUCCCCUCCCUGUUUAUGAC
AAAUGUGUGGACCCCUAGCAACCCUAACACAGUGUACCACUGCUCCGCCGUGUAU
AAUUCUGAAUUCUACUACGUGCUGUGCGCCGUGUCCGUGGUGGGAGACCCCAUCC
UGAACUCUACCUACUGGAGCGGCUCUCUGAUGAUGACCAGACUGGCUGUUAAGCC
CAAGAACAACGGCGAGAGCUACAAUCAACACCAGUUCGCCCUGCGGAACAUCGAG
AAGGGCAUGUACGACAAAGUGAUGCCCUACGGCCCUUCAGGAAUCAAGCAGGGCG
AUACCCUGUAUUUCCCCGCUGUGGGCUUCCUGGUGCGGACCGAAUUCAAGUACAA
UGACUCCAAUUGCCCCGUGGCCAAGUGUCAGUACAGCAAACCUGAAAACUGUAGA
CUGUCUAUGGGCAUCAGACCUAAUAGCCACUACAUCCUCAGAAGCGGACUCCUCA
AGUACAACCUGUCCGACGAGGAAAACUCUAAAAUUAUCUUCAUCGAGAUCAGCG
ACCAGCGCCUGUCUAUCGGAUCUCCAUCUAAGAUCUACGAUAGCCUGGGCCAACC
UGUGUUUUACCAGGCCAGCUUUAGCUGGGACACCAUGAUCAAGUUCGGAGAUGU
GCAGACAGUGAACCCCCUAGUGGUUAACUGGAGAGAUAAUACCGUGAUUAGCAG
ACCCGGCCAGUCCCAGUGUCCCAGAUUCAACACCUGCCCUGAGGUGUGCUGGGAG
GGCGUGUACAACGACGCCUUCCUGAUCGAUAGAAUCAACUGGAUCUCUGCCGGCG
UAUUUCUGGACAGCAACCAGACCGCCGAGAAUCCUGUGUUCACCGUGUUCAAGGA
UAACGAGGUGCUGUACAGAGCCCAGCUGGCCAGCGAGGACACAAACGCCCAGAAG
ACCAUCACAAACUGCUUCCUGCUGAAGAACAAGAUCUGGUGCAUCAGCCUGGUCG
AGAUCUACGACACCGGCGACAACGUGAUCCGGCCUAAGCUGUUCGCUGUGAAGAU
CCCUGAGCAGUGCACC
3) Soluble NiV-G fused with COL1A1 signal peptide mRNA
sequence (5′UTR-ORF-3′UTR-poly (A) tail)
(the ORF is underlined)
SEQ ID NO: 3
AGGCCGGCACUCUUCUGGUCCCCACAGACUCAGAGAGAACCCGCCGCCACCAUGU
UCUCUUUCGUGGACCUGCGCCUGCUGCUGCUGCUCGCUGCCACCGCCCUGCUGAC
ACACGGCCAGAACUACACCCGGAGCACCGACAACCAGGCUAUGAUCAAAGACGCC
CUGCAGAGCAUCCAACAGCAGAUCAAGGGCCUGGCCGACAAGAUCGGCACAGAAA
UCGGACCAAAGGUGUCCCUGAUCGACACGAGUUCUACAAUCACUAUCCCUGCCAA
CAUCGGCCUGCUGGGCAGCAAAAUCUCUCAGAGCACAGCCAGCAUCAACGAGAAC
GUGAACGAGAAAUGCAAGUUCACCCUGCCACCACUGAAGAUCCACGAAUGCAACA
UCAGCUGCCCUAAUCCUCUGCCCUUCAGAGAGUACAAGCCUCAGACCGAGGGUGU
GUCUAACCUGGUGGGCCUGCCGAACAACAUCUGCCUGCAAAAAACCAGCAACCAG
AUCCUGAAACCUAAGCUGAUCAGCUACACACUGCCUGUGGUGGGCCAGAGCGGCA
CCUGUAUCACAGAUCCUCUGCUGGCCAUGGAUGAAGGCUACUUCGCCUACAGCCA
UCUGGAAAAGAUCGGGAGCUGUAGCCGGGGCGUCUCCAAACAGAGAAUCAUCGG
CGUGGGCGAAGUGCUGGACAGAGGCGAUGAGGUCCCCUCCCUGUUUAUGACAAA
UGUGUGGACCCCUAGCAACCCUAACACAGUGUACCACUGCUCCGCCGUGUAUAAU
UCUGAAUUCUACUACGUGCUGUGCGCCGUGUCCGUGGUGGGAGACCCCAUCCUGA
ACUCUACCUACUGGAGCGGCUCUCUGAUGAUGACCAGACUGGCUGUUAAGCCCAA
GAACAACGGCGAGAGCUACAAUCAACACCAGUUCGCCCUGCGGAACAUCGAGAAG
GGCAUGUACGACAAAGUGAUGCCCUACGGCCCUUCAGGAAUCAAGCAGGGCGAUA
CCCUGUAUUUCCCCGCUGUGGGCUUCCUGGUGCGGACCGAAUUCAAGUACAAUGA
CUCCAAUUGCCCCGUGGCCAAGUGUCAGUACAGCAAACCUGAAAACUGUAGACUG
UCUAUGGGCAUCAGACCUAAUAGCCACUACAUCCUCAGAAGCGGACUCCUCAAGU
ACAACCUGUCCGACGAGGAAAACUCUAAAAUUAUCUUCAUCGAGAUCAGCGACCA
GCGCCUGUCUAUCGGAUCUCCAUCUAAGAUCUACGAUAGCCUGGGCCAACCUGUG
UUUUACCAGGCCAGCUUUAGCUGGGACACCAUGAUCAAGUUCGGAGAUGUGCAG
ACAGUGAACCCCCUAGUGGUUAACUGGAGAGAUAAUACCGUGAUUAGCAGACCC
GGCCAGUCCCAGUGUCCCAGAUUCAACACCUGCCCUGAGGUGUGCUGGGAGGGCG
UGUACAACGACGCCUUCCUGAUCGAUAGAAUCAACUGGAUCUCUGCCGGCGUAUU
UCUGGACAGCAACCAGACCGCCGAGAAUCCUGUGUUCACCGUGUUCAAGGAUAAC
GAGGUGCUGUACAGAGCCCAGCUGGCCAGCGAGGACACAAACGCCCAGAAGACCA
UCACAAACUGCUUCCUGCUGAAGAACAAGAUCUGGUGCAUCAGCCUGGUCGAGAU
CUACGACACCGGCGACAACGUGAUCCGGCCUAAGCUGUUCGCUGUGAAGAUCCCU
GAGCAGUGCACCUGAUAAAGCUGGAGCCUCGGUGGCCUUGCUUCUUGCCCCUUGG
GCCUCCCCCCAGCCCCUCCUCCCCUUCCUGCACCCGUACCCCCGUGGUCUUUGAAU
AAAGUCUGAGUGGGCGGCAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAA
4) Sequence of pUC57-Kan plasmid encoding Soluble NiV-G
fused with COL1A1 signal peptide mRNA(Soluble NiV-G fused
with COL1A1 signal peptide mRNA sequence is underlined)
SEQ ID NO: 4
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGTTCTC
TTTCGTGGACCTGCGCCTGCTGCTGCTGCTCGCTGCCACCGCCCTGCTGACACACGG
CCAGAACTACACCCGGAGCACCGACAACCAGGCTATGATCAAAGACGCCCTGCAGA
GCATCCAACAGCAGATCAAGGGCCTGGCCGACAAGATCGGCACAGAAATCGGACCA
AAGGTGTCCCTGATCGACACGAGTTCTACAATCACTATCCCTGCCAACATCGGCCTG
CTGGGCAGCAAAATCTCTCAGAGCACAGCCAGCATCAACGAGAACGTGAACGAGAA
ATGCAAGTTCACCCTGCCACCACTGAAGATCCACGAATGCAACATCAGCTGCCCTAA
TCCTCTGCCCTTCAGAGAGTACAAGCCTCAGACCGAGGGTGTGTCTAACCTGGTGGG
CCTGCCGAACAACATCTGCCTGCAAAAAACCAGCAACCAGATCCTGAAACCTAAGC
TGATCAGCTACACACTGCCTGTGGTGGGCCAGAGCGGCACCTGTATCACAGATCCTC
TGCTGGCCATGGATGAAGGCTACTTCGCCTACAGCCATCTGGAAAAGATCGGGAGC
TGTAGCCGGGGCGTCTCCAAACAGAGAATCATCGGCGTGGGCGAAGTGCTGGACAG
AGGCGATGAGGTCCCCTCCCTGTTTATGACAAATGTGTGGACCCCTAGCAACCCTAA
CACAGTGTACCACTGCTCCGCCGTGTATAATTCTGAATTCTACTACGTGCTGTGCGCC
GTGTCCGTGGTGGGAGACCCCATCCTGAACTCTACCTACTGGAGCGGCTCTCTGATG
ATGACCAGACTGGCTGTTAAGCCCAAGAACAACGGCGAGAGCTACAATCAACACCA
GTTCGCCCTGCGGAACATCGAGAAGGGCATGTACGACAAAGTGATGCCCTACGGCC
CTTCAGGAATCAAGCAGGGCGATACCCTGTATTTCCCCGCTGTGGGCTTCCTGGTGC
GGACCGAATTCAAGTACAATGACTCCAATTGCCCCGTGGCCAAGTGTCAGTACAGC
AAACCTGAAAACTGTAGACTGTCTATGGGCATCAGACCTAATAGCCACTACATCCTC
AGAAGCGGACTCCTCAAGTACAACCTGTCCGACGAGGAAAACTCTAAAATTATCTTC
ATCGAGATCAGCGACCAGCGCCTGTCTATCGGATCTCCATCTAAGATCTACGATAGC
CTGGGCCAACCTGTGTTTTACCAGGCCAGCTTTAGCTGGGACACCATGATCAAGTTC
GGAGATGTGCAGACAGTGAACCCCCTAGTGGTTAACTGGAGAGATAATACCGTGAT
TAGCAGACCCGGCCAGTCCCAGTGTCCCAGATTCAACACCTGCCCTGAGGTGTGCTG
GGAGGGCGTGTACAACGACGCCTTCCTGATCGATAGAATCAACTGGATCTCTGCCGG
CGTATTTCTGGACAGCAACCAGACCGCCGAGAATCCTGTGTTCACCGTGTTCAAGGA
TAACGAGGTGCTGTACAGAGCCCAGCTGGCCAGCGAGGACACAAACGCCCAGAAG
ACCATCACAAACTGCTTCCTGCTGAAGAACAAGATCTGGTGCATCAGCCTGGTCGAG
ATCTACGACACCGGCGACAACGTGATCCGGCCTAAGCTGTTCGCTGTGAAGATCCCT
GAGCAGTGCACCTGATAAAGCTGGAGCCTCGGTGGCCTTGCTTCTTGCCCCTTGGGC
CTCCCCCCAGCCCCTCCTCCCCTTCCTGCACCCGTACCCCCGTGGTCTTTGAATAAAG
TCTGAGTGGGCGGCAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAAAAAAAAAAAATGAAGAGCATCGGATCCCGGGCCCGTCGA
CTGCAGAGGCCTGCATGCAAGCTTGGTGTAATCATGGTCATAGCTGTTTCCTGTGTG
AAATTGTTATCCGCTCACAATTCCACACAACATACGAGCCGGAAGCATAAAGTGTA
AAGCCTGGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCG
CGGGGAGAGGCGGTTTGCGTATTGGGCGCTCTTCCGCTTCCTCGCTCACTGACTCGC
TGCGCTCGGTCGTTCGGCTGCGGCGAGCGGTATCAGCTCACTCAAAGGCGGTAATAC
GGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCGAAACCCG
ACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTA
ACTATCGTCTTGAGTCCAACCCGGTAAGACACGACTTATCGCCACTGGCAGCAGCCA
CTGGTAACAGGATTAGCAGAGCGAGGTATGTAGGCGGTGCTACAGAGTTCTTGAAG
TGGTGGCCTAACTACGGCTACACTAGAAGAACAGTATTTGGTATCTGCGCTCTGCTG
AAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAAAA
CTCACGTTAAGGGATTTTGGTCATGAGATTATCAAAAAGGATCTTCACCTAGATCCT
TTTAAATTAAAAATGAAGTTTTAAATCAAGCCCAATCTGAATAATGTTACAACCAAT
TAACCAATTCTGATTAGAAAAACTCATCGAGCATCAAATGAAACTGCAATTTATTCA
TATCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTATCAA
GTGAGAAATCACCATGAGTGACGACTGAATCCGGTGAGAATGGCAAAAGTTTATGC
ATTTCTTTCCAGACTTGTTCAACAGGCCAGCCATTACGCTCGTCATCAAAATCACTCG
CATCAACCAAACCGTTATTCATTCGTGATTGCGCCTGAGCGAGACGAAATACGCGAT
CGCTGTTAAAAGGACAATTACAAACAGGAATCGAATGCAACCGGCGCAGGAACACT
GCCAGCGCATCAACAATATTTTCACCTGAATCAGGATATTCTTCTAATACCTGGAAT
GCTGTTTTTCCGGGGATCGCAGTGGTGAGTAACCATGCATCATCAGGAGTACGGATA
AAATGCTTGATGGTCGGAAGAGGCATAAATTCCGTCAGCCAGTTTAGTCTGACCATC
TCATCTGTAACATCATTGGCAACGCTACCTTTGCCATGTTTCAGAAACAACTCTGGC
GCATCGGGCTTCCCATACAAGCGATAGATTGTCGCACCTGATTGCCCGACATTATCG
CGAGCCCATTTATACCCATATAAATCAGCATCCATGTTGGAATTTAATCGCGGCCTC
GACGTTTCCCGTTGAATATGGCTCATAACACCCCTTGTATTACTGTTTATGTAAGCAG
ACAGTTTTATTGTTCATGATGATATATTTTTATCTTGTGCAATGTAACATCAGAGATT
TTGAGACACGGGCCAGAGCTGCA
5) Protein sequence of full-length NiV-G
SEQ ID NO: 5
MPTESKKVRFENTASDKGKNPSKVIKSYYGTMDIKKINEGLLDSKILSAFNTVIALLGSIV
IIVMNIMIIQNYTRSTDNQAMIKDALQSIQQQIKGLADKIGTEIGPKVSLIDTSSTITIPANIG
LLGSKISQSTASINENVNEKCKFTLPPLKIHECNISCPNPLPFREYKPQTEGVSNLVGLPNN
ICLQKTSNQILKPKLISYTLPVVGQSGTCITDPLLAMDEGYFAYSHLEKIGSCSRGVSKQR
IIGVGEVLDRGDEVPSLFMTNVWTPSNPNTVYHCSAVYNSEFYYVLCAVSVVGDPILNS
TYWSGSLMMTRLAVKPKNNGESYNQHQFALRNIEKGMYDKVMPYGPSGIKQGDTLYF
PAVGFLVRTEFKYNDSNCPVAKCQYSKPENCRLSMGIRPNSHYILRSGLLKYNLSDEENS
KIIFIEISDQRLSIGSPSKIYDSLGQPVFYQASFSWDTMIKFGDVQTVNPLVVNWRDNTVIS
RPGQSQCPRFNTCPEVCWEGVYNDAFLIDRINWISAGVFLDSNQTAENPVFTVFKDNEV
LYRAQLASEDTNAQKTITNCFLLKNKIWCISLVEIYDTGDNVIRPKLFAVKIPEQCT
6) Full-length NiV-G mRNA sequence (ORF)
SEQ ID NO: 6
AUGCCUACAGAAAGCAAAAAGGUGCGGUUCGAGAACACAGCCUCUGACAAGGGA
AAGAAUCCUAGCAAGGUGAUCAAAAGCUAUUACGGCACCAUGGAUAUCAAGAAG
AUUAACGAAGGCCUGCUGGACAGCAAGAUACUCAGCGCUUUUAACACCGUGAUCG
CCCUGCUGGGCAGCAUCGUGAUCAUAGUUAUGAACAUCAUGAUUAUCCAGAACU
ACACCCGGAGCACCGACAACCAGGCUAUGAUCAAAGACGCCCUGCAGAGCAUCCA
ACAGCAGAUCAAGGGCCUGGCCGACAAGAUCGGCACAGAAAUCGGACCAAAGGUG
UCCCUGAUCGACACGAGUUCUACAAUCACUAUCCCUGCCAACAUCGGCCUGCUGG
GCAGCAAAAUCUCUCAGAGCACAGCCAGCAUCAACGAGAACGUGAACGAGAAAUG
CAAGUUCACCCUGCCACCACUGAAGAUCCACGAAUGCAACAUCAGCUGCCCUAAU
CCUCUGCCCUUCAGAGAGUACAAGCCUCAGACCGAGGGUGUGUCUAACCUGGUGG
GCCUGCCGAACAACAUCUGCCUGCAAAAAACCAGCAACCAGAUCCUGAAACCUAA
GCUGAUCAGCUACACACUGCCUGUGGUGGGCCAGAGCGGCACCUGUAUCACAGAU
CCUCUGCUGGCCAUGGAUGAAGGCUACUUCGCCUACAGCCAUCUGGAAAAGAUCG
GGAGCUGUAGCCGGGGCGUCUCCAAACAGAGAAUCAUCGGCGUGGGCGAAGUGC
UGGACAGAGGCGAUGAGGUCCCCUCCCUGUUUAUGACAAAUGUGUGGACCCCUAG
CAACCCUAACACAGUGUACCACUGCUCCGCCGUGUAUAAUUCUGAAUUCUACUAC
GUGCUGUGCGCCGUGUCCGUGGUGGGAGACCCCAUCCUGAACUCUACCUACUGGA
GCGGCUCUCUGAUGAUGACCAGACUGGCUGUUAAGCCCAAGAACAACGGCGAGAG
CUACAAUCAACACCAGUUCGCCCUGCGGAACAUCGAGAAGGGCAUGUACGACAAA
GUGAUGCCCUACGGCCCUUCAGGAAUCAAGCAGGGCGAUACCCUGUAUUUCCCCG
CUGUGGGCUUCCUGGUGCGGACCGAAUUCAAGUACAAUGACUCCAAUUGCCCCGU
GGCCAAGUGUCAGUACAGCAAACCUGAAAACUGUAGACUGUCUAUGGGCAUCAG
ACCUAAUAGCCACUACAUCCUCAGAAGCGGACUCCUCAAGUACAACCUGUCCGAC
GAGGAAAACUCUAAAAUUAUCUUCAUCGAGAUCAGCGACCAGCGCCUGUCUAUCG
GAUCUCCAUCUAAGAUCUACGAUAGCCUGGGCCAACCUGUGUUUUACCAGGCCAG
CUUUAGCUGGGACACCAUGAUCAAGUUCGGAGAUGUGCAGACAGUGAACCCCCUA
GUGGUUAACUGGAGAGAUAAUACCGUGAUUAGCAGACCCGGCCAGUCCCAGUGU
CCCAGAUUCAACACCUGCCCUGAGGUGUGCUGGGAGGGCGUGUACAACGACGCCU
UCCUGAUCGAUAGAAUCAACUGGAUCUCUGCCGGCGUAUUUCUGGACAGCAACCA
GACCGCCGAGAAUCCUGUGUUCACCGUGUUCAAGGAUAACGAGGUGCUGUACAG
AGCCCAGCUGGCCAGCGAGGACACAAACGCCCAGAAGACCAUCACAAACUGCUUC
CUGCUGAAGAACAAGAUCUGGUGCAUCAGCCUGGUCGAGAUCUACGACACCGGCG
ACAACGUGAUCCGGCCUAAGCUGUUCGCUGUGAAGAUCCCUGAGCAGUGCACC
7) Full-length NiV-G mRNA sequence (5′UTR-ORF-3′UTR-poly
(A) tail) (the ORF is underlined)
SEQ ID NO: 7
AGGCCGGCACUCUUCUGGUCCCCACAGACUCAGAGAGAACCCGCCGCCACCAUGC
CUACAGAAAGCAAAAAGGUGCGGUUCGAGAACACAGCCUCUGACAAGGGAAAGA
AUCCUAGCAAGGUGAUCAAAAGCUAUUACGGCACCAUGGAUAUCAAGAAGAUUA
ACGAAGGCCUGCUGGACAGCAAGAUACUCAGCGCUUUUAACACCGUGAUCGCCCU
GCUGGGCAGCAUCGUGAUCAUAGUUAUGAACAUCAUGAUUAUCCAGAACUACAC
CCGGAGCACCGACAACCAGGCUAUGAUCAAAGACGCCCUGCAGAGCAUCCAACAG
CAGAUCAAGGGCCUGGCCGACAAGAUCGGCACAGAAAUCGGACCAAAGGUGUCCC
UGAUCGACACGAGUUCUACAAUCACUAUCCCUGCCAACAUCGGCCUGCUGGGCAG
CAAAAUCUCUCAGAGCACAGCCAGCAUCAACGAGAACGUGAACGAGAAAUGCAAG
UUCACCCUGCCACCACUGAAGAUCCACGAAUGCAACAUCAGCUGCCCUAAUCCUC
UGCCCUUCAGAGAGUACAAGCCUCAGACCGAGGGUGUGUCUAACCUGGUGGGCCU
GCCGAACAACAUCUGCCUGCAAAAAACCAGCAACCAGAUCCUGAAACCUAAGCUG
AUCAGCUACACACUGCCUGUGGUGGGCCAGAGCGGCACCUGUAUCACAGAUCCUC
UGCUGGCCAUGGAUGAAGGCUACUUCGCCUACAGCCAUCUGGAAAAGAUCGGGA
GCUGUAGCCGGGGCGUCUCCAAACAGAGAAUCAUCGGCGUGGGCGAAGUGCUGG
ACAGAGGCGAUGAGGUCCCCUCCCUGUUUAUGACAAAUGUGUGGACCCCUAGCAA
CCCUAACACAGUGUACCACUGCUCCGCCGUGUAUAAUUCUGAAUUCUACUACGUG
CUGUGCGCCGUGUCCGUGGUGGGAGACCCCAUCCUGAACUCUACCUACUGGAGCG
GCUCUCUGAUGAUGACCAGACUGGCUGUUAAGCCCAAGAACAACGGCGAGAGCUA
CAAUCAACACCAGUUCGCCCUGCGGAACAUCGAGAAGGGCAUGUACGACAAAGUG
AUGCCCUACGGCCCUUCAGGAAUCAAGCAGGGCGAUACCCUGUAUUUCCCCGCUG
UGGGCUUCCUGGUGCGGACCGAAUUCAAGUACAAUGACUCCAAUUGCCCCGUGGC
CAAGUGUCAGUACAGCAAACCUGAAAACUGUAGACUGUCUAUGGGCAUCAGACC
UAAUAGCCACUACAUCCUCAGAAGCGGACUCCUCAAGUACAACCUGUCCGACGAG
GAAAACUCUAAAAUUAUCUUCAUCGAGAUCAGCGACCAGCGCCUGUCUAUCGGAU
CUCCAUCUAAGAUCUACGAUAGCCUGGGCCAACCUGUGUUUUACCAGGCCAGCUU
UAGCUGGGACACCAUGAUCAAGUUCGGAGAUGUGCAGACAGUGAACCCCCUAGU
GGUUAACUGGAGAGAUAAUACCGUGAUUAGCAGACCCGGCCAGUCCCAGUGUCCC
AGAUUCAACACCUGCCCUGAGGUGUGCUGGGAGGGCGUGUACAACGACGCCUUCC
UGAUCGAUAGAAUCAACUGGAUCUCUGCCGGCGUAUUUCUGGACAGCAACCAGAC
CGCCGAGAAUCCUGUGUUCACCGUGUUCAAGGAUAACGAGGUGCUGUACAGAGCC
CAGCUGGCCAGCGAGGACACAAACGCCCAGAAGACCAUCACAAACUGCUUCCUGC
UGAAGAACAAGAUCUGGUGCAUCAGCCUGGUCGAGAUCUACGACACCGGCGACAA
CGUGAUCCGGCCUAAGCUGUUCGCUGUGAAGAUCCCUGAGCAGUGCACCUGAUAA
AGCUGGAGCCUCGGUGGCCUUGCUUCUUGCCCCUUGGGCCUCCCCCCAGCCCCUC
CUCCCCUUCCUGCACCCGUACCCCCGUGGUCUUUGAAUAAAGUCUGAGUGGGCGG
CAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAA
8) Sequence of pUC57-Kan plasmid encoding Full-length
NiV-G (Full-length NiV-G mRNA sequence is underlined)
SEQ ID NO: 8
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGCCTAC
AGAAAGCAAAAAGGTGCGGTTCGAGAACACAGCCTCTGACAAGGGAAAGAATCCT
AGCAAGGTGATCAAAAGCTATTACGGCACCATGGATATCAAGAAGATTAACGAAGG
CCTGCTGGACAGCAAGATACTCAGCGCTTTTAACACCGTGATCGCCCTGCTGGGCAG
CATCGTGATCATAGTTATGAACATCATGATTATCCAGAACTACACCCGGAGCACCGA
CAACCAGGCTATGATCAAAGACGCCCTGCAGAGCATCCAACAGCAGATCAAGGGCC
TGGCCGACAAGATCGGCACAGAAATCGGACCAAAGGTGTCCCTGATCGACACGAGT
TCTACAATCACTATCCCTGCCAACATCGGCCTGCTGGGCAGCAAAATCTCTCAGAGC
ACAGCCAGCATCAACGAGAACGTGAACGAGAAATGCAAGTTCACCCTGCCACCACT
GAAGATCCACGAATGCAACATCAGCTGCCCTAATCCTCTGCCCTTCAGAGAGTACAA
GCCTCAGACCGAGGGTGTGTCTAACCTGGTGGGCCTGCCGAACAACATCTGCCTGCA
AAAAACCAGCAACCAGATCCTGAAACCTAAGCTGATCAGCTACACACTGCCTGTGG
TGGGCCAGAGCGGCACCTGTATCACAGATCCTCTGCTGGCCATGGATGAAGGCTACT
TCGCCTACAGCCATCTGGAAAAGATCGGGAGCTGTAGCCGGGGCGTCTCCAAACAG
AGAATCATCGGCGTGGGCGAAGTGCTGGACAGAGGCGATGAGGTCCCCTCCCTGTT
TATGACAAATGTGTGGACCCCTAGCAACCCTAACACAGTGTACCACTGCTCCGCCGT
GTATAATTCTGAATTCTACTACGTGCTGTGCGCCGTGTCCGTGGTGGGAGACCCCAT
CCTGAACTCTACCTACTGGAGCGGCTCTCTGATGATGACCAGACTGGCTGTTAAGCC
CAAGAACAACGGCGAGAGCTACAATCAACACCAGTTCGCCCTGCGGAACATCGAGA
AGGGCATGTACGACAAAGTGATGCCCTACGGCCCTTCAGGAATCAAGCAGGGCGAT
ACCCTGTATTTCCCCGCTGTGGGCTTCCTGGTGCGGACCGAATTCAAGTACAATGAC
TCCAATTGCCCCGTGGCCAAGTGTCAGTACAGCAAACCTGAAAACTGTAGACTGTCT
ATGGGCATCAGACCTAATAGCCACTACATCCTCAGAAGCGGACTCCTCAAGTACAA
CCTGTCCGACGAGGAAAACTCTAAAATTATCTTCATCGAGATCAGCGACCAGCGCCT
GTCTATCGGATCTCCATCTAAGATCTACGATAGCCTGGGCCAACCTGTGTTTTACCA
GGCCAGCTTTAGCTGGGACACCATGATCAAGTTCGGAGATGTGCAGACAGTGAACC
CCCTAGTGGTTAACTGGAGAGATAATACCGTGATTAGCAGACCCGGCCAGTCCCAGT
GTCCCAGATTCAACACCTGCCCTGAGGTGTGCTGGGAGGGCGTGTACAACGACGCC
TTCCTGATCGATAGAATCAACTGGATCTCTGCCGGCGTATTTCTGGACAGCAACCAG
ACCGCCGAGAATCCTGTGTTCACCGTGTTCAAGGATAACGAGGTGCTGTACAGAGCC
CAGCTGGCCAGCGAGGACACAAACGCCCAGAAGACCATCACAAACTGCTTCCTGCT
GAAGAACAAGATCTGGTGCATCAGCCTGGTCGAGATCTACGACACCGGCGACAACG
TGATCCGGCCTAAGCTGTTCGCTGTGAAGATCCCTGAGCAGTGCACCTGATAAAGCT
GGAGCCTCGGTGGCCTTGCTTCTTGCCCCTTGGGCCTCCCCCCAGCCCCTCCTCCCCT
TCCTGCACCCGTACCCCCGTGGTCTTTGAATAAAGTCTGAGTGGGCGGCAAAAAAAA
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAATGAAGAGCATCGGATCCCGGGCCCGTCGACTGCAGAGGCCTGCATGCAAGCT
TGGTGTAATCATGGTCATAGCTGTTTCCTGTGTGAAATTGTTATCCGCTCACAATTCC
ACACAACATACGAGCCGGAAGCATAAAGTGTAAAGCCTGGGGTGCCTAATGAGTGA
GCTAACTCACATTAATTGCGTTGCGCTCACTGCCCGCTTTCCAGTCGGGAAACCTGT
CGTGCCAGCTGCATTAATGAATCGGCCAACGCGCGGGGAGAGGCGGTTTGCGTATT
GGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGA
GGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAG
TTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCAGTGGAACGAAAACTCACGTTAAGGGATTTTGGTCATGA
GATTATCAAAAAGGATCTTCACCTAGATCCTTTTAAATTAAAAATGAAGTTTTAAAT
CAAGCCCAATCTGAATAATGTTACAACCAATTAACCAATTCTGATTAGAAAAACTCA
TCGAGCATCAAATGAAACTGCAATTTATTCATATCAGGATTATCAATACCATATTTTT
GAAAAAGCCGTTTCTGTAATGAAGGAGAAAACTCACCGAGGCAGTTCCATAGGATG
GCAAGATCCTGGTATCGGTCTGCGATTCCGACTCGTCCAACATCAATACAACCTATT
AATTTCCCCTCGTCAAAAATAAGGTTATCAAGTGAGAAATCACCATGAGTGACGACT
GAATCCGGTGAGAATGGCAAAAGTTTATGCATTTCTTTCCAGACTTGTTCAACAGGC
CAGCCATTACGCTCGTCATCAAAATCACTCGCATCAACCAAACCGTTATTCATTCGT
GATTGCGCCTGAGCGAGACGAAATACGCGATCGCTGTTAAAAGGACAATTACAAAC
AGGAATCGAATGCAACCGGCGCAGGAACACTGCCAGCGCATCAACAATATTTTCAC
CTGAATCAGGATATTCTTCTAATACCTGGAATGCTGTTTTTCCGGGGATCGCAGTGG
TGAGTAACCATGCATCATCAGGAGTACGGATAAAATGCTTGATGGTCGGAAGAGGC
ATAAATTCCGTCAGCCAGTTTAGTCTGACCATCTCATCTGTAACATCATTGGCAACG
CTACCTTTGCCATGTTTCAGAAACAACTCTGGCGCATCGGGCTTCCCATACAAGCGA
TAGATTGTCGCACCTGATTGCCCGACATTATCGCGAGCCCATTTATACCCATATAAA
TCAGCATCCATGTTGGAATTTAATCGCGGCCTCGACGTTTCCCGTTGAATATGGCTC
ATAACACCCCTTGTATTACTGTTTATGTAAGCAGACAGTTTTATTGTTCATGATGATA
TATTTTTATCTTGTGCAATGTAACATCAGAGATTTTGAGACACGGGCCAGAGCTGCA
9) Protein sequence of the full-length NiV-F
SEQ ID NO: 9
MAVILNKRYYSNLLLLILMISECSVGILHYEKLSKIGLVKGITRKYKIKSNPLTKDIVIKMI
PNVSNMSQCTGSVMENYKTRLNGILTPIKGALEIYKNNTHDLVGDVRLAGVIMAGVAIG
IATAAQITAGVALYEAMKNADNINKLKSSIESTNEAVVKLQETAEKTVYVLTALQDYIN
TNLVPTIDKISCKQTELSLDLALSKYLSDLLFVFGPNLQDPVSNSMTIQAISQAFGGNYET
LLRTLGYATEDFDDLLESDSITGQIIYVDLSGYYIIVRVYFPILTEIQQAYIQELLPVSFNND
NSEWISIVPNFILVRNTLISNIEIGFCLITKRSVICNQDYATPMTNNMRECLTGSTEKCPRE
LVVSSHVPRFALSNGVLFANCISVTCQCQTTGRAISQSGEQTLLMIDNTTCPTAVLGNVII
SLGKYLGSVNYNSEGIAIGPPVFTDKVDISSQISSMNQSLQQSKDYIKEAQRLLDTVNPSL
ISMLSMIILYVLSIASLCIGLITFISFIIVEKKRNTYSRLEDRRVRPTSSGDLYYIGT
10) Full-length NiV-F mRNA sequence (ORF)
SEQ ID NO: 10
AUGGCCGUGAUCCUGAACAAGCGGUACUACUCUAAUCUGCUGCUGCUGAUCCUGA
UGAUCAGCGAGUGCAGCGUGGGCAUUCUCCACUACGAGAAGCUGUCCAAAAUCGG
CCUGGUGAAAGGCAUCACAAGAAAGUACAAGAUCAAAAGCAACCCUCUGACCAAG
GACAUCGUGAUUAAGAUGAUCCCUAAUGUGAGCAAUAUGAGCCAGUGCACCGGU
AGCGUGAUGGAAAACUACAAGACCAGACUGAACGGCAUCCUGACCCCUAUCAAGG
GCGCCCUGGAAAUCUAUAAGAACAACACACAUGACCUGGUGGGAGAUGUGCGGC
UGGCUGGCGUGAUUAUGGCCGGCGUGGCCAUCGGAAUCGCCACAGCCGCCCAGAU
CACCGCCGGCGUCGCCCUGUACGAGGCCAUGAAAAACGCCGAUAAUAUCAACAAG
CUGAAGUCUAGCAUCGAGUCUACAAACGAGGCCGUGGUGAAGCUGCAGGAGACA
GCAGAGAAGACCGUGUACGUGCUGACCGCCCUGCAGGACUACAUUAACACCAAUC
UGGUGCCUACCAUCGACAAGAUCUCAUGUAAGCAGACCGAGCUGUCUCUGGAUCU
GGCCCUGAGCAAAUAUCUGUCUGAUCUGCUGUUCGUGUUCGGCCCUAACCUGCAA
GACCCUGUUUCCAAUUCCAUGACAAUCCAAGCCAUAAGCCAGGCCUUCGGCGGCA
AUUACGAAACCCUGCUUAGAACCCUGGGCUACGCCACAGAGGACUUCGACGACCU
GCUGGAGAGCGACAGCAUCACCGGACAGAUCAUCUACGUUGAUCUGUCCGGCUAC
UAUAUCAUCGUGAGAGUGUACUUCCCCAUCCUGACUGAGAUCCAGCAGGCUUACA
UACAGGAGCUGCUGCCAGUGAGCUUCAACAACGACAAUUCUGAAUGGAUCAGCA
UCGUGCCCAACUUCAUCCUGGUGCGGAACACCCUCAUCAGCAACAUCGAGAUCGG
AUUUUGCCUGAUCACCAAGCGGAGCGUGAUCUGCAACCAGGAUUACGCCACACCU
AUGACCAACAAUAUGCGGGAAUGCCUGACAGGAUCUACCGAGAAGUGCCCCAGAG
AACUGGUCGUGUCCAGCCACGUGCCAAGAUUCGCCCUGUCUAACGGCGUGCUGUU
UGCCAACUGCAUCUCUGUGACCUGUCAGUGUCAGACAACCGGCAGAGCCAUCAGC
CAGAGCGGCGAGCAGACCCUGCUGAUGAUCGACAACACCACAUGCCCUACAGCUG
UUCUGGGCAACGUGAUCAUUUCCCUGGGAAAGUACCUGGGCUCUGUGAACUAUA
ACAGCGAAGGCAUCGCGAUUGGACCUCCUGUGUUCACCGACAAGGUGGACAUCAG
CAGCCAAAUCAGUAGCAUGAACCAGAGCCUGCAGCAGAGCAAGGACUACAUUAAG
GAAGCUCAGAGACUGCUGGACACCGUGAACCCCAGCCUGAUCUCUAUGCUGUCUA
UGAUCAUCCUGUACGUGCUGAGCAUCGCCUCCCUUUGUAUCGGCCUGAUCACCUU
CAUCAGCUUUAUCAUCGUGGAAAAAAAGAGAAACACCUACAGUAGACUGGAAGA
UAGAAGGGUGCGCCCCACAAGCAGCGGCGACCUGUACUACAUAGGCACC
11) Full-length NiV-F mRNA sequence (5′UTR-ORF-3′UTR-poly
(A) tail) (the ORF is underlined)
SEQ ID NO: 11
AGGCCGGCACUCUUCUGGUCCCCACAGACUCAGAGAGAACCCGCCGCCACCAUGG
CCGUGAUCCUGAACAAGCGGUACUACUCUAAUCUGCUGCUGCUGAUCCUGAUGAU
CAGCGAGUGCAGCGUGGGCAUUCUCCACUACGAGAAGCUGUCCAAAAUCGGCCUG
GUGAAAGGCAUCACAAGAAAGUACAAGAUCAAAAGCAACCCUCUGACCAAGGAC
AUCGUGAUUAAGAUGAUCCCUAAUGUGAGCAAUAUGAGCCAGUGCACCGGUAGC
GUGAUGGAAAACUACAAGACCAGACUGAACGGCAUCCUGACCCCUAUCAAGGGCG
CCCUGGAAAUCUAUAAGAACAACACACAUGACCUGGUGGGAGAUGUGCGGCUGG
CUGGCGUGAUUAUGGCCGGCGUGGCCAUCGGAAUCGCCACAGCCGCCCAGAUCAC
CGCCGGCGUCGCCCUGUACGAGGCCAUGAAAAACGCCGAUAAUAUCAACAAGCUG
AAGUCUAGCAUCGAGUCUACAAACGAGGCCGUGGUGAAGCUGCAGGAGACAGCA
GAGAAGACCGUGUACGUGCUGACCGCCCUGCAGGACUACAUUAACACCAAUCUGG
UGCCUACCAUCGACAAGAUCUCAUGUAAGCAGACCGAGCUGUCUCUGGAUCUGGC
CCUGAGCAAAUAUCUGUCUGAUCUGCUGUUCGUGUUCGGCCCUAACCUGCAAGAC
CCUGUUUCCAAUUCCAUGACAAUCCAAGCCAUAAGCCAGGCCUUCGGCGGCAAUU
ACGAAACCCUGCUUAGAACCCUGGGCUACGCCACAGAGGACUUCGACGACCUGCU
GGAGAGCGACAGCAUCACCGGACAGAUCAUCUACGUUGAUCUGUCCGGCUACUAU
AUCAUCGUGAGAGUGUACUUCCCCAUCCUGACUGAGAUCCAGCAGGCUUACAUAC
AGGAGCUGCUGCCAGUGAGCUUCAACAACGACAAUUCUGAAUGGAUCAGCAUCG
UGCCCAACUUCAUCCUGGUGCGGAACACCCUCAUCAGCAACAUCGAGAUCGGAUU
UUGCCUGAUCACCAAGCGGAGCGUGAUCUGCAACCAGGAUUACGCCACACCUAUG
ACCAACAAUAUGCGGGAAUGCCUGACAGGAUCUACCGAGAAGUGCCCCAGAGAAC
UGGUCGUGUCCAGCCACGUGCCAAGAUUCGCCCUGUCUAACGGCGUGCUGUUUGC
CAACUGCAUCUCUGUGACCUGUCAGUGUCAGACAACCGGCAGAGCCAUCAGCCAG
AGCGGCGAGCAGACCCUGCUGAUGAUCGACAACACCACAUGCCCUACAGCUGUUC
UGGGCAACGUGAUCAUUUCCCUGGGAAAGUACCUGGGCUCUGUGAACUAUAACA
GCGAAGGCAUCGCGAUUGGACCUCCUGUGUUCACCGACAAGGUGGACAUCAGCAG
CCAAAUCAGUAGCAUGAACCAGAGCCUGCAGCAGAGCAAGGACUACAUUAAGGA
AGCUCAGAGACUGCUGGACACCGUGAACCCCAGCCUGAUCUCUAUGCUGUCUAUG
AUCAUCCUGUACGUGCUGAGCAUCGCCUCCCUUUGUAUCGGCCUGAUCACCUUCA
UCAGCUUUAUCAUCGUGGAAAAAAAGAGAAACACCUACAGUAGACUGGAAGAUA
GAAGGGUGCGCCCCACAAGCAGCGGCGACCUGUACUACAUAGGCACCUGACUCGA
GUAAGCUGGAGCCUCGGUGGCCAUGCUUCUUGCCCCUUGGGCCUCCCCCCAGCCC
CUCCUCCCCUUCCUGCACCCGUACCCCCGUGGUCUUUGAAUAAAGUCUGAGUGGG
CGGCAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAAAAAA
12) Sequence of pUC57-Kan plasmid encoding Full-length
NiV-F (Full-length NiV-F mRNA sequence is underlined)
SEQ ID NO: 12
TCGCGCGTTTCGGTGATGACGGTGAAAACCTCTGACACATGCAGCTCCCGGAGACG
GTCACAGCTTGTCTGTAAGCGGATGCCGGGAGCAGACAAGCCCGTCAGGGCGCGTC
AGCGGGTGTTGGCGGGTGTCGGGGCTGGCTTAACTATGCGGCATCAGAGCAGATTG
TACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATC
GGTGCGGGCCTCTTCGCTATTACGCCAGCTGGCGAAAGGGGGATGTGCTGCAAGGC
GATTAAGTTGGGTAACGCCAGGGTTTTCCCAGTCACGACGTTGTAAAACGACGGCCA
GAGAATTCGAGCTCGGTACCTCGCGAATACATCTAGATTAATACGACTCACTATAAG
GCCGGCACTCTTCTGGTCCCCACAGACTCAGAGAGAACCCGCCGCCACCATGGCCGT
GATCCTGAACAAGCGGTACTACTCTAATCTGCTGCTGCTGATCCTGATGATCAGCGA
GTGCAGCGTGGGCATTCTCCACTACGAGAAGCTGTCCAAAATCGGCCTGGTGAAAG
GCATCACAAGAAAGTACAAGATCAAAAGCAACCCTCTGACCAAGGACATCGTGATT
AAGATGATCCCTAATGTGAGCAATATGAGCCAGTGCACCGGTAGCGTGATGGAAAA
CTACAAGACCAGACTGAACGGCATCCTGACCCCTATCAAGGGCGCCCTGGAAATCT
ATAAGAACAACACACATGACCTGGTGGGAGATGTGCGGCTGGCTGGCGTGATTATG
GCCGGCGTGGCCATCGGAATCGCCACAGCCGCCCAGATCACCGCCGGCGTCGCCCT
GTACGAGGCCATGAAAAACGCCGATAATATCAACAAGCTGAAGTCTAGCATCGAGT
CTACAAACGAGGCCGTGGTGAAGCTGCAGGAGACAGCAGAGAAGACCGTGTACGTG
CTGACCGCCCTGCAGGACTACATTAACACCAATCTGGTGCCTACCATCGACAAGATC
TCATGTAAGCAGACCGAGCTGTCTCTGGATCTGGCCCTGAGCAAATATCTGTCTGAT
CTGCTGTTCGTGTTCGGCCCTAACCTGCAAGACCCTGTTTCCAATTCCATGACAATCC
AAGCCATAAGCCAGGCCTTCGGCGGCAATTACGAAACCCTGCTTAGAACCCTGGGC
TACGCCACAGAGGACTTCGACGACCTGCTGGAGAGCGACAGCATCACCGGACAGAT
CATCTACGTTGATCTGTCCGGCTACTATATCATCGTGAGAGTGTACTTCCCCATCCTG
ACTGAGATCCAGCAGGCTTACATACAGGAGCTGCTGCCAGTGAGCTTCAACAACGA
CAATTCTGAATGGATCAGCATCGTGCCCAACTTCATCCTGGTGCGGAACACCCTCAT
CAGCAACATCGAGATCGGATTTTGCCTGATCACCAAGCGGAGCGTGATCTGCAACC
AGGATTACGCCACACCTATGACCAACAATATGCGGGAATGCCTGACAGGATCTACC
GAGAAGTGCCCCAGAGAACTGGTCGTGTCCAGCCACGTGCCAAGATTCGCCCTGTCT
AACGGCGTGCTGTTTGCCAACTGCATCTCTGTGACCTGTCAGTGTCAGACAACCGGC
AGAGCCATCAGCCAGAGCGGCGAGCAGACCCTGCTGATGATCGACAACACCACATG
CCCTACAGCTGTTCTGGGCAACGTGATCATTTCCCTGGGAAAGTACCTGGGCTCTGT
GAACTATAACAGCGAAGGCATCGCGATTGGACCTCCTGTGTTCACCGACAAGGTGG
ACATCAGCAGCCAAATCAGTAGCATGAACCAGAGCCTGCAGCAGAGCAAGGACTAC
ATTAAGGAAGCTCAGAGACTGCTGGACACCGTGAACCCCAGCCTGATCTCTATGCTG
TCTATGATCATCCTGTACGTGCTGAGCATCGCCTCCCTTTGTATCGGCCTGATCACCT
TCATCAGCTTTATCATCGTGGAAAAAAAGAGAAACACCTACAGTAGACTGGAAGAT
AGAAGGGTGCGCCCCACAAGCAGCGGCGACCTGTACTACATAGGCACCTGACTCGA
GTAAGCTGGAGCCTCGGTGGCCATGCTTCTTGCCCCTTGGGCCTCCCCCCAGCCCCT
CCTCCCCTTCCTGCACCCGTACCCCCGTGGTCTTTGAATAAAGTCTGAGTGGGCGGC
AAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAA
AAAAAAAAAAAATGAAGAGCATCGGATCCCGGGCCCGTCGACTGCAGAGGCCTGC
ATGCAAGCTTGGTGTAATCATGGTCATAGCTGTTTCCTGTGTGAAATTGTTATCCGCT
CACAATTCCACACAACATACGAGCCGGAAGCATAAAGTGTAAAGCCTGGGGTGCCT
AATGAGTGAGCTAACTCACATTAATTGCGTTGCGCTCACTGCCCGCTTTCCAGTCGG
GAAACCTGTCGTGCCAGCTGCATTAATGAATCGGCCAACGCGCGGGGAGAGGCGGT
TTGCGTATTGGGCGCTCTTCCGCTTCCTCGCTCACTGACTCGCTGCGCTCGGTCGTTC
GGCTGCGGCGAGCGGTATCAGCTCACTCAAAGGCGGTAATACGGTTATCCACAGAA
TCAGGGGATAACGCAGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCC
TTTGATCTTTTCTACGGGGTCTGACGCTCAGTGGAACGAAAACTCACGTTAAGGGAT
TTTGGTCATGAGATTATCAAAAAGGATCTTCACCTAGATCCTTTTAAATTAAAAATG
AAGTTTTAAATCAAGCCCAATCTGAATAATGTTACAACCAATTAACCAATTCTGATT
AGAAAAACTCATCGAGCATCAAATGAAACTGCAATTTATTCATATCAGGATTATCAA
TACCATATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAACATCAA
TACAACCTATTAATTTCCCCTCGTCAAAAATAAGGTTATCAAGTGAGAAATCACCAT
GAGTGACGACTGAATCCGGTGAGAATGGCAAAAGTTTATGCATTTCTTTCCAGACTT
GTTCAACAGGCCAGCCATTACGCTCGTCATCAAAATCACTCGCATCAACCAAACCGT
TATTCATTCGTGATTGCGCCTGAGCGAGACGAAATACGCGATCGCTGTTAAAAGGAC
AATTACAAACAGGAATCGAATGCAACCGGCGCAGGAACACTGCCAGCGCATCAACA
ATATTTTCACCTGAATCAGGATATTCTTCTAATACCTGGAATGCTGTTTTTCCGGGGA
TCGCAGTGGTGAGTAACCATGCATCATCAGGAGTACGGATAAAATGCTTGATGGTC
GGAAGAGGCATAAATTCCGTCAGCCAGTTTAGTCTGACCATCTCATCTGTAACATCA
TTGGCAACGCTACCTTTGCCATGTTTCAGAAACAACTCTGGCGCATCGGGCTTCCCA
TACAAGCGATAGATTGTCGCACCTGATTGCCCGACATTATCGCGAGCCCATTTATAC
CCATATAAATCAGCATCCATGTTGGAATTTAATCGCGGCCTCGACGTTTCCCGTTGA
ATATGGCTCATAACACCCCTTGTATTACTGTTTATGTAAGCAGACAGTTTTATTGTTC
ATGATGATATATTTTTATCTTGTGCAATGTAACATCAGAGATTTTGAGACACGGGCC
AGAGCTGCA

### EXAMPLES

### Example 1—DNA Templates Used for In Vitro Transcription and Protein Expression

DNA template sequence for mRNA in vitro transcription (IVT) is consisted of T7 promoter, 5′ untranslated region (UTR), open reading frame (ORF) of Nipah virus glycoprotein (NiV-G) and fusion protein (NiV-F) modified from MCL-18-H-1088 strain (GenBank. MH523642.1), 3′UTR and 120 bases of poly adenine (polyA). 5′UTR and 3′UTR are from human hemoglobin subunit alpha 1 (HBA1) mRNA (GenBank. NM_000558.5). For soluble form of glycoprotein (NiV-G sol), the amino terminal and transmembrane domain was removed from the full-length glycoprotein sequence, resulting in a potential secreted form (ΔMet1-Ile70). The signal peptide from collagen alpha1, COL1A1 (ColSP, MFSFVDLRLLLLLAATALLTHG, GenBank: Z74615.1) was added to the N-terminus of full length and soluble glycoprotein ORF to facilitate its secretion into media. The initial NiV-G DNA was synthesized and subcloned into pUC57-Kan vector by GenScript (Piscataway, NJ). The constructs of NiV-G_sol, ColSP-NiV-G and ColSP-NiV-G_sol were subsequently made through PCRs and subcloning into the NiV-G construct and verified the sequences via Sanger sequencing (Azenta, Inc). The NiV-F DNA was synthesized from Twist BioScience and subsequently subcloned into pUC57-Kan vector. The constructs of NiV-F with (ColSP-NiV-F) and without ColSP (NiV-F) were made through PCRs along with FLAG-tagged ones (ColSP-NiV-F-FLAG and NiV-F-FLAG) to ensure the protein expression and detection.

The Sequence of pUC57-Kan plasmid encoding soluble NiV-G fused with COL1A1 signal peptide mRNA was shown in SEQ ID NO: 4. The sequence of pUC57-Kan plasmid encoding full-length NiV-G mRNA was shown in SEQ ID NO: 8. The sequence of pUC57-Kan plasmid encoding full-length NiV-F mRNA is shown in SEQ ID NO: 12.

### Example 2—In Vitro Transcription (IVT)

The plasmid vector was linearized by restriction enzyme, BspQI (New England Biolabs) for each NiV forms. N1-Methylpseudouridine (m1ψ) was purchase from BOC Sciences (Shirley, NY). IVT condition is followed by manufacture's recommendation (TranscriptAid T7 High Yield Transcription Kit, ThermoFisher) as below:
ATP/CTP/GTP/m1ψTP: 5 mM each
SmartCap (SC101, ST Pharm): 4 mM
Linear template DNA: 1 ug of plasmid or 0.5 ug of PCR product
T7 RNA polymerase enzyme mix: 2 ul

IVT was carried out in 20 ul reaction incubated at 37 C for 2 hours. The template DNA is removed by 2 units of DNase I (Invitrogen) treated at 37 C for 15 min followed by a column purification (Monarch RNA Cleanup Kit, New England Biolabs).

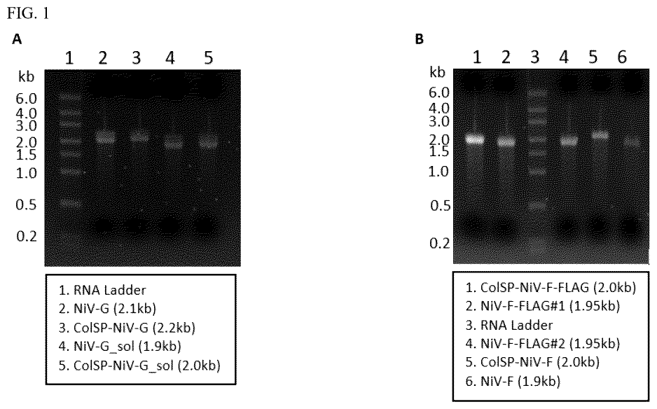

The IVT products of four NiV-G constructs (A) and four NiV-F constructs (B) were analyzed by agarose gel, and ~2knt long mRNAs for these four mRNAs were detected. After IVT from DNA templates of NiV-G mRNAs (A) and NiV-F mRNAs (B), 100 ng of mRNAs were run on 1% agarose of E-GEL EX in E-Gel Power Snap Electrophoresis Device (ThermoFisher) (one of three independent IVT products). See FIG. 1.

### Example 3—Transfection

1 ug of mRNAs (synthesized in triplicates) were individually transfected into 293FT cells (Invitrogen) in 12 well plate using Lipofectamine MesseangerMax (Invitrogen), 2 ul at 1:2 ratio according to the manufacturer's protocol. Samples were collected from both media and cells after 24 hours of transfection. Cell lysates were prepared in NP-40 lysis buffer (150 mM sodium chloride/1% NP-40/50 mM Tris pH8.0). As a transfection control, 0.1 ug of EGFP mRNA (L-7601, TriLink) was co-transfected.

### Example 4—Western Blot

Rabbit anti-Nipah Virus Glycoprotein antibody (#NIV11-S) was purchased from Alpha Diagnostic (San Antonio, TX). Detection of protein was using HRP-conjugated secondary antibodies (Jackson ImmunoResearch, West Grove, PA) and SuperSignal West Pico Plus Chemiluminescent Substrate (Thermo Scientific). GAPDH was detected as a loading control by a HRP-conjugated mouse monoclonal antibody (sc-47724, Santa Cruz Biotechnology). EGFP, a transfection control, was detected with a mouse monoclonal antibody (sc-9996, Santa Cruz Biotechnology). For NiV-F protein detection, anti-FLAG-tag monoclonal antibody (GenScript) and anti-Nipah virus F F1 rabbit antibody (Absolute Antibody, Wilton, UK) were purchased.

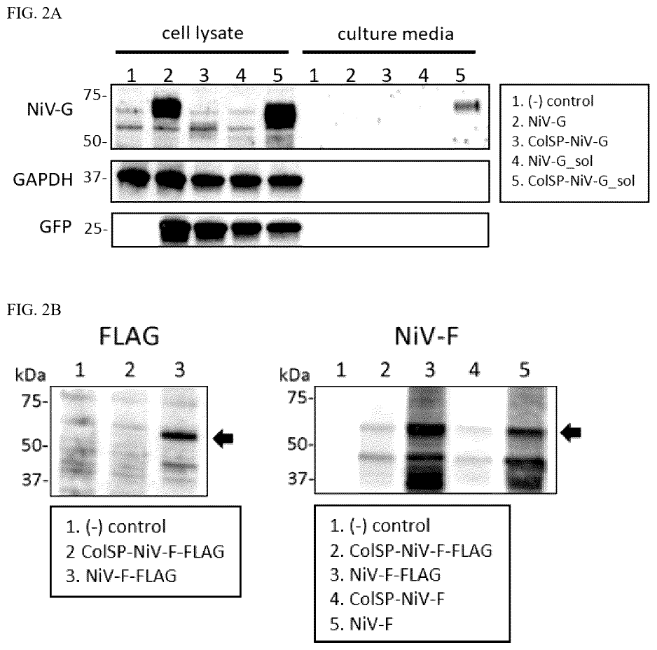

As shown in FIG. 2A, NiV-G protein levels were determined by Western blots. 293FT cells were individually transfected with 1 ug of the four NiV-G mRNAs. Both cell lysates and culture media were collected at 24 hour post transfection and subjected to Western Blot with NiV-G specific antibody. The GFP mRNA was co-transfected for a mRNA transfection control, and non-transfected 293FT cells were used as a negative control. GAPDH was used for a loading control. As shown in FIG. 2B, NiV-F proteins with or without ColSP were detected with FLAG-tag antibody and NiV-F specific antibody in cell lysates, showing 60 kDa full-length NiV-F protein (arrow) and smaller cleaved ones. No-transfected cell lysate or media were used as a negative control ((−) control).

Western blots were conducted from collected samples from both media for detecting any secreted NiV-G proteins and cell lysates for detecting intracellular/un-secreted NiV-G proteins. As shown in FIG. 2A, wild type NiV-G proteins without COL1A1 signal peptide (lane 2) and the soluble NiV-G with COL1A1 signal peptide (lane 5) were detected in cell lysates. But, only the soluble NiV-G proteins with COL1A1 signal peptide were detected in the media. Since NiV-G and ColSP-NiV-G_sol are well expressed compared to ColSP-NiV-G and NiV-G_sol, we selected NiV-G and ColSP-NiV-G_sol as our mRNA vaccine candidate. For NiV-F proteins, in FIG. 2B, cell lysates were probed with FLAG antibody and further confirmed with NiV-F specific antibody to detect NiV-F proteins. Similar to NiV-G, wild type NiV-F was better expressed than ColSP-NiV-F in the full-length form, selecting wild type full-length NiV-F as another mRNA vaccine candidate.

### Example 5—Immunogenicity Study

This study was designed to test the immunogenicity in mice of the Nipah virus vaccine composition of the present disclosure (e.g., Nipah virus vaccine compositions (1) and (4)).

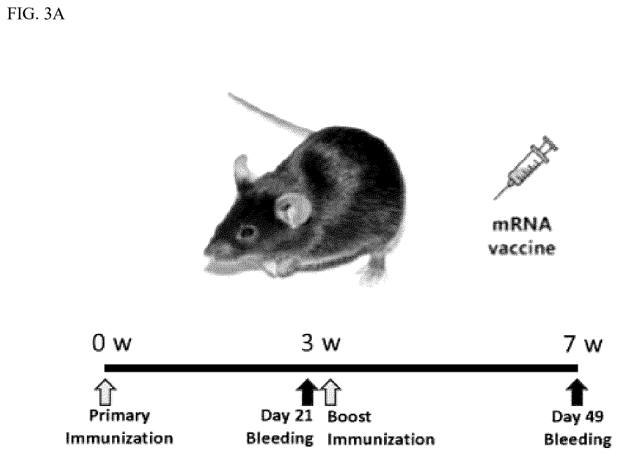

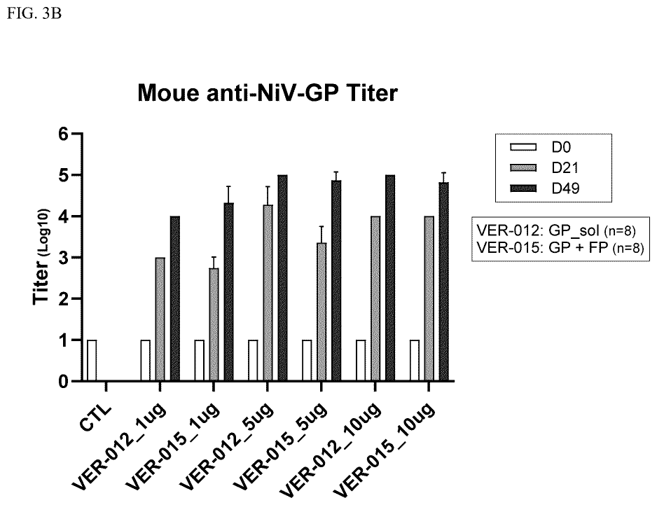

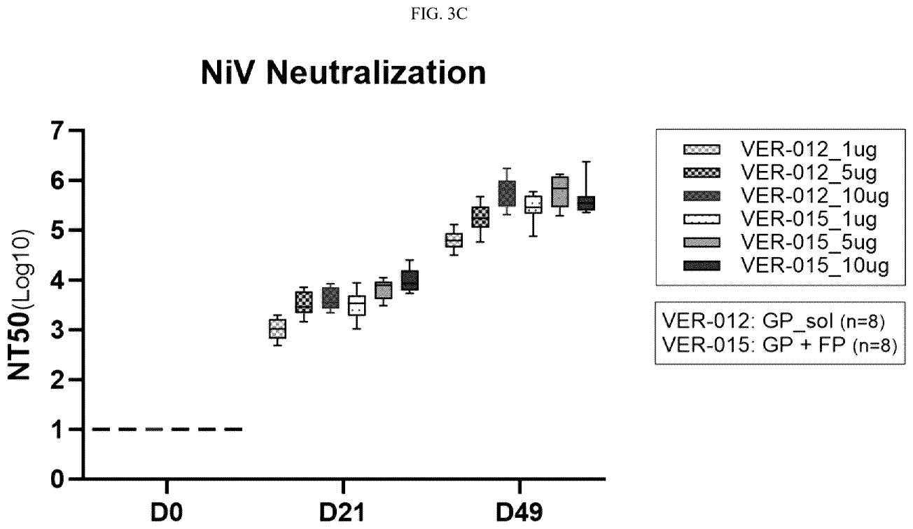

Mice were immunized intramuscularly (IM) with the Nipah virus vaccine composition of the present disclosure. The vaccine composition of the present disclosure is chemically modified or unmodified. A total of two immunizations were given at 3-week intervals (i.e., at weeks 0, and 3), and sera were collected after each immunization. See FIG. 3A. Serum antibody titers against soluble NiV-G fused with COL1A1 signal peptide and full-length NiV-G were determined by Mouse anti-Nipah Virus Glycoprotein IgG ELISA Kit (NIV-025, Alpha Diagnostic). Collectively, both formulations of mRNA vaccine (VER-012 with soluble NiV-G (Nipah virus vaccine composition (1)) and VER-015 with full lengths of NiV-G and NiV-F (Nipah virus vaccine composition (4)) showed high titer of IgG antibodies against NiV-G in dose dependent manner as shown in FIG. 3B. Sera collected from each mouse were also used for in vitro protection assay against Nipha virus as shown in FIG. 3C of NiV neutralization. Both vaccine formulations displayed a strong neutralization activity against Nipha virus in dose dependent manner. Overall, the full length formulation of NiV-G and NiV-F (VER-015) performed better in neutralizing Nipha virus compared with the soluble NiV-G (VER-012).

### Example 6—Additional Immunogenicity Study

This study is designed to test the immunogenicity in mice of the Nipah virus vaccine composition of the present disclosure (e.g., Nipah virus vaccine compositions (2) and (3)).

Mice are immunized intramuscularly (IM) with the Nipah virus vaccine composition of the present disclosure. The vaccine composition of the present disclosure is chemically modified or unmodified. A total of two immunizations were given at 3-week intervals (i.e., at weeks 0, and 3), and sera are collected after each immunization.

Both formulations of mRNA vaccine (VER-013 with the full length of NiV-G (Nipah virus vaccine composition (2)) and VER-014 with the full length of NiV-F (Nipah virus vaccine composition (3)) are expected to show high titer of IgG antibodies against Nipah virus in dose dependent manner. Sera collected from each mouse are also used for in vitro protection assay against Nipha virus of NiV neutralization. Both vaccine formulations are expected to display a strong neutralization activity against Nipha virus.

1. A Nipah virus vaccine composition comprising
a messenger ribonucleic acid (mRNA) comprising an open reading frame (ORF) encoding soluble Nipah virus glycoprotein (soluble NiV-G) fused with human collagen type I alpha 1 (COL1A1) signal peptide.

2. The Nipah virus vaccine composition according to claim 1, wherein the soluble NiV-G fused with COL1A1 signal peptide has an amino acid sequence of SEQ ID NO: 1.

3. The Nipah virus vaccine composition according to claim 1, wherein the ORF encoding soluble NiV-G fused with COL1A1 signal peptide has a nucleotide sequence of SEQ ID NO: 2.

4. The Nipah virus vaccine composition according to claim 1, wherein the mRNA comprising the ORF encoding soluble NiV-G fused with COL1A1 signal peptide further comprises a 5′ untranslated region (UTR), a 3′ UTR, and a poly (A) tail so as to have the following structure:
5′UTR-ORF encoding soluble NiV-G fused with COL1A1 signal peptide-3′UTR-poly (A) tail, and
wherein the ORF encoding soluble NiV-G fused with COL1A1 signal peptide has a nucleotide sequence of SEQ ID NO: 2.

5. The Nipah virus vaccine composition according to claim 4, wherein the poly (A) tail has a length of 50-250 nucleotides.

6. The Nipah virus vaccine composition according to claim 4, wherein the mRNA having the structure of 5′UTR-ORF encoding soluble NiV-G fused with COL1A1 signal peptide-3′UTR-poly (A) tail has a nucleotide sequence of SEQ ID NO: 3.

7. The Nipah virus vaccine composition according to claim 4, wherein the mRNA having the structure of 5′UTR-ORF encoding soluble NiV-G fused with COL1A1 signal peptide-3′UTR-poly (A) tail has a nucleotide sequence having at least 80% identity to SEQ ID NO: 3.

8. A method of inducing immune response against Nipah virus comprising:
administering an effective amount of the Nipah virus vaccine composition according to claim 1 to a subject in need thereof.

9. A Nipah virus vaccine composition comprising
a messenger ribonucleic acid (mRNA) comprising an open reading frame (ORF) encoding full-length Nipah virus glycoprotein (full-length NiV-G).

10. The Nipah virus vaccine composition according to claim 9, wherein the full-length NiV-G has an amino acid sequence of SEQ ID NO: 5.

11. The Nipah virus vaccine composition according to claim 9, wherein the ORF encoding full-length NiV-G has a nucleotide sequence of SEQ ID NO: 6.

12. The Nipah virus vaccine composition according to claim 9, wherein the mRNA comprising the ORF encoding full-length NiV-G further comprises a 5′ untranslated region (UTR), a 3′ UTR, and a poly (A) tail so as to have the following structure:
5′UTR-ORF encoding full-length NiV-G-3′UTR-poly (A) tail, and
wherein the ORF encoding full-length NiV-G has a nucleotide sequence of SEQ ID NO: 6.

13. The Nipah virus vaccine composition according to claim 12, wherein the poly (A) tail has a length of 50-250 nucleotides.

14. The Nipah virus vaccine composition according to claim 12, wherein the mRNA having the structure of 5′UTR-ORF encoding full-length NiV-G-3′UTR-poly (A) tail has a nucleotide sequence of SEQ ID NO: 7.

15. The Nipah virus vaccine composition according to claim 12, wherein the mRNA having the structure of 5′UTR-ORF encoding full-length NiV-G-3′UTR-poly (A) tail has a nucleotide sequence having at least 80% identity to SEQ ID NO: 7.

16. A method of inducing immune response against Nipah virus comprising:
administering an effective amount of the Nipah virus vaccine composition according to claim 9 to a subject in need thereof.

17. A Nipah virus vaccine composition comprising
a messenger ribonucleic acid (mRNA) comprising an open reading frame (ORF) encoding full-length Nipah virus fusion protein (full-length NiV-F).

18. The Nipah virus vaccine composition according to claim 17, wherein the full-length NiV-F has an amino acid sequence of SEQ ID NO: 9.

19. The Nipah virus vaccine composition according to claim 17, wherein the ORF encoding full-length NiV-F has a nucleotide sequence of SEQ ID NO: 10.

20. The Nipah virus vaccine composition according to claim 17, wherein the mRNA comprising the ORF encoding full-length NiV-F further comprises a 5′ untranslated region (UTR), a 3′ UTR, and a poly (A) tail so as to have the following structure:
5′UTR-ORF encoding full-length NiV-F-3′UTR-poly (A) tail, and
wherein the ORF encoding full-length NiV-F has a nucleotide sequence of SEQ ID NO: 10.

21. The Nipah virus vaccine composition according to claim 20, wherein the poly (A) tail has a length of 50-250 nucleotides.

22. The Nipah virus vaccine composition according to claim 20, wherein the mRNA having the structure of 5′UTR-ORF encoding full-length NiV-F-3′UTR-poly (A) tail has a nucleotide sequence of SEQ ID NO: 11.

23. The Nipah virus vaccine composition according to claim 20, wherein the mRNA having the structure of 5′UTR-ORF encoding full-length NiV-F-3′UTR-poly (A) tail has a nucleotide sequence having at least 80% identity to SEQ ID NO: 11.

24. A method of inducing immune response against Nipah virus comprising:
administering an effective amount of the Nipah virus vaccine composition according to claim 17 to a subject in need thereof.

25. A Nipah virus vaccine composition comprising
a messenger ribonucleic acid (mRNA) comprising an open reading frame (ORF) encoding full-length Nipah virus glycoprotein (full-length NiV-G), and
a mRNA comprising an ORF encoding full-length Nipah virus fusion protein (full-length NiV-F).

26. The Nipah virus vaccine composition according to claim 25, wherein the full-length NiV-G has an amino acid sequence of SEQ ID NO: 5, and
wherein the full-length NiV-F has an amino acid sequence of SEQ ID NO: 9.

27. The Nipah virus vaccine composition according to claim 25, wherein the ORF encoding full-length NiV-G has a nucleotide sequence of SEQ ID NO: 6, and
wherein the ORF encoding full-length NiV-F has a nucleotide sequence of SEQ ID NO: 10.

28. The Nipah virus vaccine composition according to claim 25, wherein the mRNA comprising the ORF encoding full-length NiV-G further comprises a 5′ untranslated region (UTR), a 3′ UTR, and a poly (A) tail so as to have the following structure:
5′UTR-ORF encoding full-length NiV-G-3′UTR-poly (A) tail, and
the ORF encoding full-length NiV-G has a nucleotide sequence of SEQ ID NO: 6, and
wherein the mRNA comprising the ORF encoding full-length NiV-F further comprises a 5′ UTR, a 3′ UTR, and a poly (A) tail so as to have the following structure:
5′UTR-ORF encoding full-length NiV-F-3′UTR-poly (A) tail, and
the ORF encoding full-length NiV-F has a nucleotide sequence of SEQ ID NO: 10.

29. The Nipah virus vaccine composition according to claim 28, wherein the poly (A) tail has a length of 50-250 nucleotides.

30. The Nipah virus vaccine composition according to claim 28, wherein the mRNA having the structure of 5′UTR-ORF encoding full-length NiV-G-3′UTR-poly (A) tail has a nucleotide sequence of SEQ ID NO: 7, and
wherein the mRNA having the structure of 5′UTR-ORF encoding full-length NiV-F-3′UTR-poly (A) tail has a nucleotide sequence of SEQ ID NO: 11.

31. The Nipah virus vaccine composition according to claim 28, wherein the mRNA having the structure of 5′UTR-ORF encoding full-length NiV-G-3′UTR-poly (A) tail has a nucleotide sequence having at least 80% identity to SEQ ID NO: 7, and
wherein the mRNA having the structure of 5′UTR-ORF encoding full-length NiV-F-3′UTR-poly (A) tail has a nucleotide sequence having at least 80% identity to SEQ ID NO: 11.

32. A method of inducing immune response against Nipah virus comprising:
administering an effective amount of the Nipah virus vaccine composition according to claim 25 to a subject in need thereof.
